(19) United States
(12) Patent Application Publication
(10) Pub. No.: US 2021/0244698 A1
(43) Pub. Date: Aug. 12, 2021

(54) DIFFUSION ENHANCING COMPOUNDS AND THEIR USE WITH THROMBECTOMY AND EMBOLECTOMY AND OTHER VASCULAR DISEASE PROCEDURES

(71) Applicant: DIFFUSION PHARMACEUTICALS LLC, Charlottesville, VA (US)

(72) Inventors: David G. KALERGIS, Charlottesville, VA (US); Thomas BYRNE, Alexandria, VA (US)

(73) Assignee: DIFFUSION PHARMACEUTICALS LLC, Charlottesville, VA (US)

(21) Appl. No.: 17/264,411

(22) PCT Filed: Jul. 30, 2019
(86) PCT No.: PCT/US2019/044232

§ 371 Date: Jan. 29, 2021

Related U.S. Application Data
(60) Provisional application No. 62/712,012, filed on Jul. 30, 2018.

(51) Int. Cl. A61K 31/202; A61K 47/40; A61K 45/06; A61K 9/00; A61B 18/20

(52) U.S. Cl. CPC A61K 31/202; A61K 47/40; A61B 2018/0041; A61K 9/0019; A61B 18/20; A61K 45/06

(57) ABSTRACT

The subject invention relates to novel methods for the rapid treatment of disorders resulting from thrombosis or embolism such as a myocardial infraction or stroke. Specifically, the invention relates to diffusion enhancing compounds and their use with embolectomy and thrombectomy, or other procedures for the treatment of ischemia.

This application claims priority to U.S. Provisional Application No. 62/712,012 filed Jul. 30, 2018, which is hereby incorporated by reference in its entirety.

### FIELD OF THE INVENTION

The subject invention relates to novel methods for the rapid treatment of disorders resulting from thrombosis or embolism such as a myocardial infarction or stroke (brain infarction). Specifically, the invention relates to diffusion enhancing compounds and their use with embolectomy and thrombectomy, or other procedures for the treatment of ischemia.

### BACKGROUND OF THE INVENTION

Thrombosis is the formation or presence of a blood clot in a blood vessel. The vessel may be any vein or artery as, for example, in a deep vein thrombosis or a coronary (artery) thrombosis. The clot itself is termed a thrombus. A thrombus is the inappropriate activation of the hemostatic process in an uninjured or slightly injured vessel. A thrombus in a large blood vessel (mural thrombus) will decrease blood flow through that vessel. In a small blood vessel (occlusive thrombus), blood flow may be completely cut-off resulting in death of tissue supplied by that vessel (infarction). If a thrombus dislodges and becomes free-floating, it is termed an embolus. The most common type of embolus is a blood clot generated by thrombosis which has broken off and is then transported in the blood stream.

An embolus is an abnormal mass of material (which can be solid, liquid or gas but is typically a clot) that is carried in the blood stream from one part of the circulation to another causing a blockage (occlusion) of a blood vessel that leads to lack of oxygen supply (ischemia) and finally infarction of tissue downstream of the embolus. The penumbra is the area surrounding an ischemic event such as thrombotic or embolic stroke. Immediately following the event, blood flow and therefore oxygen transport is reduced locally, leading to hypoxia of the cells near the location of the original insult.

There are two areas where emboli can form and therefore impact tissue: i) arterial emboli form in the left side of the heart or the main arteries—they impact body tissues but not the lungs, commonly in the brain and the small vessels in the upper and lower limbs, and ii) venous emboli arise in veins (for example emboli which form from deep venous thrombosis or DVT) and these impact the lungs (pulmonary embolism).

Some of the conditions which elevate risk of blood clots developing include atrial fibrillation (a form of cardiac arrhythmia), heart valve replacement, a recent heart attack, extended periods of inactivity (see deep venous thrombosis below), and genetic or disease-related deficiencies in the blood's clotting abilities.

Blood clot prevention and treatment reduces the risk of stroke, heart attack and pulmonary embolism. Heparin and warfarin are often used to inhibit the formation and growth of existing thrombi; they are able to decrease blood coagulation by inhibiting vitamin K epoxide reductase, an enzyme needed to form mature clotting factors.

Embolectomy and Thrombectomy

Thrombectomy and embolectomy are emergency procedures. The terms embolectomy and thrombectomy are sometimes used interchangeably, but there are some differences between the two. To understand how a thrombectomy or embolectomy is performed, it is important to understand why they are done.

Thrombosis (occlusion) is the formation or presence of a blood clot in a blood vessel. The vessel may be any vein or artery as, for example, in a deep vein thrombosis or a coronary (artery) thrombosis. Due to various factors like disease, blood clots can form in the blood vessels.

A thrombus is usually a solid-mass stationary clot. The most common type of embolus is when part or all of that clot is dislodged and begins to travel through the circulatory system. These clots can pose serious and even fatal risks.

An embolism is the lodging of an embolus, a blockage-causing piece of material, inside a blood vessel. The embolus may be a blood clot, a fat globule (causing fat embolism), a bubble of air or other gas (causing gas embolism), or foreign material. An embolism can cause partial or total blockage of blood flow in the affected vessel. Such a blockage (a vascular occlusion) may affect a part of the body distant to the origin of the embolus. An embolism in which the embolus is a piece of thrombus is called a thromboembolism.

When an artery is obstructed by a thrombus or embolus, it is called a thromboembolism or embolism. Types of embolisms include:
Thromboembolism—formation in a blood vessel by a blood clot that has become dislodged from another site and carried through the bloodstream
Cholesterol embolism—Blockage of a blood vessel as the result of atherosclerotic plaque
Fat embolism—Blockage of a blood vessel caused by fat or bone fractures
Air embolism—Obstruction of a blood vessel by gaseous matter, such as an air bubble
Septic embolism—A bacteria-containing pus blockage of a blood vessel
Tissue embolism—A blockage of a blood vessel formed by natural tissues within the body
Foreign body embolism—A blockage of a blood vessel that wasn't naturally produced by the body
Amniotic fluid embolism—An obstruction of a blood vessel formed by amniotic fluid, fetal cells, hair or other debris that have entered the mother's bloodstream

A thrombectomy is the removal of a thrombus and an embolectomy is the removal of an embolus. As used herein, the terms thrombectomy and embolectomy do not include thrombolysis.

Types of Embolectomy and Thrombectomy

There are two main types of embolectomy and thrombectomy, depending on the blood vessel that needs treatment and the severity of the condition. These are:

Catheter-Based Procedures

Catheter-based procedures involve passing a small tube through a tiny incision into the clot site. Special instruments are used to remove the clot by balloon embolectomy or aspiration embolectomy. Balloon embolectomy is done by inserting a catheter with a balloon attached at the end into the vein. An aspiration embolectomy is performed by using suction to remove the thrombus from the vein.

Open Surgery

Open surgery involves making a larger incision in the area of the blood clot through the blood vessel to remove it. Open surgery is less common but is sometimes the best choice for emergencies to save an organ or in other cases.

Thrombolysis

Thrombolysis is the dissolving of a clot using medication. The only FDA approved treatment for ischemic strokes is tissue plasminogen activator (tPA, also known as IV rtPA, given through an IV in the arm). tPA works by dissolving the clot and improving blood flow to the part of the brain being deprived of blood flow. If administered within 3 hours (and up to 4.5 hours in certain eligible patients), tPA may improve the chances of recovering from a stroke. A significant number of stroke victims don't get to the hospital in time for tPA treatment.

Tissue plasminogen activator is a protein thrombolytic agent (clot-busting drug). It is approved for use in certain patients having a heart attack or stroke. The drug can dissolve blood clots, which cause most heart attacks and strokes. tPA is the only drug approved by the U.S. Food and Drug Administration for the acute (urgent) treatment of ischemic stroke. Specifically, it is approved for the treatment of ischemic stroke in the first three hours after the start of symptoms.

If given promptly, tPA can significantly reduce the effects of ischemic stroke and reduce permanent disability. However, a time delay in starting tPA treatment often occurs because, when a patient presents with stroke-like symptoms, it is not immediately apparent whether the stroke has been caused by blood clots (ischemic stroke) or by a ruptured blood vessel (hemorrhagic stroke). tPA can only be given for ischemic strokes; therefore, the type of stroke must be determined before tPA is administered.

Although over 80% of all strokes are ischemic strokes, tPA or any thrombolytic, cannot be given immediately since it is possible that it could cause the hemorrhagic strokes to produce even worse effects. Determining whether a given patient has suffered a hemorrhagic or ischemic stroke is a time-consuming diagnosis which stands as a “gate” to immediate treatment. That, coupled with the fact that tPA must be given within 3 hours of the first symptoms (4.5 in certain circumstances), has resulted in only a small fraction of stroke patients receiving tPA.

Ischemic Stroke

For a given isolated blood vessel, blood flow to the brain tissue can be hampered in two ways: i) the vessel clogs within (ischemic stroke), or ii) the vessel ruptures, causing blood to leak into the brain (hemorrhagic stroke). Ischemic stroke occurs when an artery to the brain is blocked. The brain depends on its arteries to bring fresh blood from the heart and lungs. The blood carries oxygen and nutrients to the brain, and takes away carbon dioxide and cellular waste. If an artery is blocked, the brain cells (neurons) cannot make enough energy and will eventually stop working. If the artery remains blocked for more than a few minutes, the brain cells may die. This is why immediate medical treatment is critical.

Ischemic stroke accounts for about 87 percent of all cases of stroke (the rest are hemorrhagic). The underlying condition for ischemic stroke is the development of fatty deposits lining the vessel walls. This condition is called atherosclerosis.

Ischemic stroke can be caused by several different kinds of diseases. The most common problem is narrowing of the arteries in the neck or head. This is most often caused by atherosclerosis, or gradual cholesterol deposition. If the arteries become too narrow, blood cells may collect and form blood clots. These blood clots can block the artery where they are formed (thrombosis), or can dislodge and become trapped in arteries closer to the brain (embolism). Another cause of stroke is blood clots in the heart, which can occur as a result of irregular heartbeat (for example, atrial fibrillation), heart attack, or abnormalities of the heart valves. While these are the most common causes of ischemic stroke, there are many other possible causes. Examples include use of street drugs, traumatic injury to the blood vessels of the neck, or disorders of blood clotting.

Ischemic stroke can be divided into two main types: thrombotic and embolic. A thrombotic stroke occurs when diseased or damaged cerebral arteries become blocked by the formation of a blood clot within the brain. Clinically referred to as cerebral thrombosis or cerebral infarction, this type of event is responsible for almost 50 percent of all strokes. Cerebral thrombosis can also be divided into an additional two categories that correlate to the location of the blockage within the brain: large-vessel thrombosis and small-vessel thrombosis. Large-vessel thrombosis is the term used when the blockage is in one of the brain's larger blood-supplying arteries such as the carotid or middle cerebral, while small-vessel thrombosis involves one (or more) of the brain's smaller, yet deeper, penetrating arteries. This latter type of stroke is also called a lacunar stroke.

An embolic stroke or cerebral embolism is also caused by a clot within an artery, but in this case the clot (or emboli) forms somewhere other than in the brain itself. Often from the heart, these emboli will travel in the bloodstream until they become lodged and cannot travel any farther. This naturally restricts the flow of blood to the brain and results in near-immediate physical and neurological deficits.

Acute ischemic stroke is a potentially devastating disease that goes untreated in the vast majority of patients. Acute ischemic stroke is estimated to affect more than 700,000 patients each year in the USA and 15 million worldwide. New methods that can reduce the clinical deficits associated with acute ischemic stroke are needed.

The mainstay of treatment for ischemic stroke has long been tissue plasminogen activator, or tPA a clot-busting drug approved by the Food and Drug Administration in 1996 that must be given intravenously within 4.5 hours to be effective.

Endovascular Procedures for Ischemic Stroke

Mechanical thrombectomy is a procedure in which trained doctors try removing a large blood clot by sending a wired-caged device called a stent retriever, to the site of the blocked blood vessel in the brain. To remove the brain clot, doctors thread a catheter through an artery in the groin up to the blocked artery in the brain. The stent opens and grabs the clot, allowing doctors to remove the stent with the trapped clot. Special suction tubes may also be used. The procedure is advantageously done within six hours of acute stroke symptoms.

The American Heart Association is giving stent retrievers, or “stentrievers” its strongest recommendation after a string of recent studies found they improve the odds that certain patients will survive and function normally again.

Stent retrievers are being used at hospitals across the United States, including all 90 comprehensive stroke centers. The FDA cleared two stent-retrieval devices in 2012: Solitaire, made by Minneapolis-based Medtronic, and Trevo, made by Stryker Corporation of Kalamazoo, Mich. These newer clot-snagging stents are safer and more effective than older devices that resemble a corkscrew.

Four devices have been approved by the FDA for the endovascular treatment of acute ischemic stroke, as follows:

Merci Retriever (Concentric Medical, Mountain View, Calif.): Corkscrew-shaped device that captures and engages clots

Penumbra System (Penumbra, Alameda, Calif.): Employs both aspiration and extraction

Solitaire FR Revascularization Device (Covidien, Dublin, Ireland): Stent-retriever system; combines the ability to restore blood flow and retrieve clot

Trevo (Concentric Medical, Mountain View, Calif.): Stent-retriever system

Successful recanalization occurred in 12 of 28 patients in the Mechanical Embolus Retrieval in Cerebral Ischemia (MERCI) 1 pilot trial, a study of the Merci Retrieval System. In a second MERCI study, recanalization was achieved in 48% of patients in whom the device was deployed. Clot was successfully retrieved from all major cerebral arteries; however, the recanalization rate for the middle cerebral artery was lowest.

The Multi MERCI trial used the newer-generation Concentric retrieval device (L5). Recanalization was demonstrated in approximately 55% of patients who did not receive t-PA and in 68% of those to whom t-PA was given. Seventy-three percent of patients who failed intravenous t-PA therapy had recanalization following mechanical embolectomy. On the basis of these results, the FDA cleared the use of the MERCI device in patients who are either ineligible for or who have failed intravenous fibrinolytics.

In a trial of the Penumbra System in 23 patients who presented within 8 hours of symptom onset, revascularization to a Thrombolysis in Myocardial Infarction (TIMI) grade of 2 or 3 was accomplished in all 21 treated vessels. Vessel tortuosity prevented access by the device in 3 patients.

More recent trials of the stent-retriever systems demonstrated superiority in reperfusion over the original Merci systems. In the Solitaire Flow Restoration Device Versus the Merci Retriever in Patients with Acute Ischemic Stroke (SWIFT) study, which enrolled 113 subjects with moderate or severe strokes within 8 hours after symptom onset, the Solitaire FR system demonstrated successful revascularization (TIMI 2-3 flow) in 61% of patients, compared with 24% of patients treated with the Merci system. Patients in the Solitaire FR group also had a higher rate of good 90-day clinical outcomes than did those in the Merci group (58% versus 33%, respectively).

A similar study, the Trevo Versus Merci Retrievers for Thrombectomy Revascularisation of Large Vessel Occlusions in Acute Ischemic Stroke (TREVO 2) trial, reported successful reperfusion (TIMI 2-3 flow) in 86% of patients using the Trevor stent retriever, compared with 60% in the Merci group. The rate of good clinical outcomes at 90 days was also higher in the Trevo group than in the Merci group (40% vs 22%, respectively).

The 2017 American Heart Association/American Stroke Association guidelines for the emergency treatment of patients with acute ischemic stroke extend the time limit on mechanical clot removal from 6 hours to up to 24 hours in select patients. The new guidelines recommend thrombectomy in eligible patients 6 to 16 hours after a stroke.

Heart Attack/Myocardial Infarction

A heart attack or myocardial infarction (MI) develops when the amount of oxygen supplied to the heart is less than the amount needed by the heart. As early as 1912, a physician named Herrick discovered that myocardial infarction (heart attack) is caused by coronary artery thrombosis. Thrombosis is usually associated with dissection (a tear in the inner wall) of the affected artery, which occurs as a result of pre-existing vascular disease. Dissection leads to the activation of platelet aggregation, and results in clot (thrombus) formation. When a temporary or prolonged occlusion of the vessel leads to an insufficient amount of blood and oxygen reaching the relevant section of the heart muscle, a heart attack occurs.

Ever since the advent of appropriate drug-treatment in the 1980s, antiplatelet therapy and thrombolytic therapy have formed an integral part of the treatment for acute heart attack. Recanalization of the coronary arteries is a superior treatment strategy when compared to purely drug-based therapy, a fact that has been known since the mid-1990s, and has led to the introduction of 24-hour emergency care for patients with acute heart attack.

A STEMI or ST-elevation myocardial infarction is caused by a sudden complete (100%) blockage of a heart artery (coronary artery). A non-STEMI is usually caused by a severely narrowed artery but the artery is usually not completely blocked. The diagnosis is initially made by an electrocardiogram (ECG or EKG).

Endovascular Procedures for Heart Attack

Percutaneous Coronary Intervention (PCI, formerly known as angioplasty with stent) is a non-surgical procedure that uses a catheter (a thin flexible tube) to place a small structure called a stent to open up blood vessels in the heart that have been narrowed by plaque buildup, a condition known as atherosclerosis. PCI improves blood flow, thus decreasing heart-related chest pain (angina).
A catheter is inserted into the blood vessels either in the groin or in the arm.
Using a special type of X-ray called fluoroscopy, the catheter is threaded through the blood vessels into the heart where the coronary artery is narrowed.
When the tip is in place, a balloon tip covered with a stent is inflated.
The balloon tip compresses the plaque and expands the stent.
Once the plaque is compressed and the stent is in place, the balloon is deflated and withdrawn.
The stent stays in the artery, holding it open.

Percutaneous coronary intervention began as percutaneous transluminal coronary angioplasty (PTCA), a term still found in the literature.

Pulmonary Embolism

Pulmonary embolism is the sudden blockage of a major blood vessel (artery) in the lung, usually by a blood clot. In most cases, the clots are small and are not deadly, but they can damage the lung. But if the clot is large and stops blood flow to the lung, it can be deadly.

Surgical or catheter embolectomy is performed in patients with pulmonary embolism (formed from venous embolisms). Embolectomy is used for patients with persisting shock despite supportive care and who have an absolute contraindication for thrombolytic therapy. Catheter embolectomy may be a life-saving procedure in severe pulmonary embolism.

Carotenoids are a class of hydrocarbons consisting of isoprenoid units. The backbone of the molecule consists of conjugated carbon-carbon double and single bonds, and can have pendant groups. Carotenoids such as crocetin and trans sodium crocetinate (TSC) are known to increase the diffusivity of oxygen in water.

U.S. Pat. No. 6,060,511 relates to trans sodium crocetinate (TSC) and its uses. The patent covers various uses of TSC such as improving oxygen diffusivity and treatment of hemorrhagic shock.

U.S. Pat. No. 7,759,506 relates to synthesis methods for making bipolar trans carotenoids (BTC), including bipolar trans carotenoid salts (BTCS), and methods of using them.

U.S. Pat. No. 8,030,350 relates to improved BTC synthesis methods and novel uses of the BTC.

U.S. Pat. No. 8,293,804 relates to the use of bipolar trans carotenoids as a pretreatment and in the treatment of peripheral vascular disease.

U.S. Pat. No. 8,206,751 relates to a new class of therapeutics that enhance small molecule diffusion.

U.S. application Ser. No. 12/801,726 relates to diffusion enhancing compounds and their use alone or with thrombolytics.

### SUMMARY OF THE INVENTION

The subject invention relates to a method of treating a patient suspected of having an embolism or thrombosis, or infarction, comprising: a) administering a diffusion enhancing compound to said patient as soon as possible after the first embolism or thrombosis symptoms, b) determining whether said patient has an embolism or thrombosis, and if so determined, c) performing an embolectomy or thrombectomy on said patient. The embolectomy or thrombectomy is a catheter based endovascular procedure, or a surgical embolectomy or thrombectomy. In the endovascular procedures, a mesh stent device is often placed in the blood vessel to support it and keep it open. Catheter based thrombectomy can involve a balloon catheter (Fogarty catheter) that is inserted into the blood vessel and through a clot. The balloon is then inflated and the clot is then extracted from the vessel. Catheters can involve the aspiration/suction of blood clots. Another catheter system uses saline jets that dislodge and remove the clot using the Bernoulli effect. Other types of thrombectomy or embolectomy catheters disrupt the clot mechanically using clot retriever, snare-like device, laser based device or ultrasound device. Optionally, the method includes the administration of a thrombolytic agent (e.g. tPA) after determination that the patient has an embolism or thrombosis. In another embodiment, step c) is replace with catheter directed thrombolysis.

As used herein, the phrase “determining” means receipt of a definitive external manifestation of the presence of the condition being discussed.

The invention also relates to a method of treating a patient having (no step b)), or suspected of having (include step b)), an ischemic stroke comprising:
a) administering a diffusion enhancing compound to said patient within 6 (advantageously 2 or less) hours of the first stroke symptoms,
b) determining whether said patient has an ischemic stroke, and if so determined (diagnosed as ischemic stroke),
c) performing a catheter based endovascular embolectomy or thrombectomy on said patient.
The embolectomy or thrombectomy is typically performed with: clot retrievers including stent retrievers or devices with a balloon that can pull out a clot; aspiration/suction devices including rheolytic devices; ultrasound based devices; laser based devices; or snare-like devices. Optionally, the method further comprises the administration of a thrombolytic agent after step b) where stroke is determined to be ischemic. In another embodiment, step c) is replace with catheter directed thrombolysis.

In another embodiment, the invention relates to a method of treating a patient having a stroke where it is unknown whether the stroke is an ischemic stroke or a hemorrhagic stroke comprising:
a) administering a diffusion enhancing compound to said patient within 6 (advantageously 2) hours of the first stroke symptoms,
b) determining whether the stroke is an ischemic stroke, and if so determined,
c) performing a catheter based endovascular embolectomy or thrombectomy (e.g. using a stent retriever or aspiration device) on said patient.

The embolectomy or thrombectomy is typically performed with: clot retrievers including stent retrievers or devices with a balloon that can pull out a clot; aspiration/suction devices including rheolytic devices; ultrasound based devices; laser based devices; or snare-like devices. Optionally, the method further comprises the administration of a thrombolytic agent after step b), where stroke is determined to be ischemic. In another embodiment, step c) is replace with catheter directed thrombolysis.

In a further embodiment, the invention relates to a method of treating a patient having a hemorrhagic stroke comprising:
a) administering a diffusion enhancing compound to said patient within 6 (advantageously 2) hours of the first stroke symptoms,
b) inserting a coil or clipping the artery at the site of the brain hemorrhage in said patient.

In a still further embodiment, the invention relates to a method of treating a patient having (no step b)), or suspected of having (include step b)), a myocardial infarction (MI) comprising:
a) administering a diffusion enhancing compound to said patient within 6 (advantageously 2) hours of the first MI symptoms,
b) determining whether said patient has a myocardial infarction, and if so determined,
c) performing percutaneous coronary intervention (PCI) on said patient.

Optionally, the clot causing the MI is removed (e.g. using aspiration or laser) prior to performing PCI. Optionally, step c) includes rotational or laser atherectomy, and/or brachytherapy. Optionally, a thrombolytic agent such as tPA is administered after determination that the patient has a myocardial infarction. In another embodiment, step c) is replace with catheter directed thrombolysis.

In another embodiment, the invention includes a method of treating a patient having (no step b)), or suspected of having (include step b)), a myocardial infarction comprising:
a) administering a diffusion enhancing compound to said patient within 6 (advantageously 2) hours of the first MI symptoms,
b) determining whether said patient has a myocardial infarction, and if so determined,
c) performing a catheter based endovascular thrombectomy.

Typically, the thrombectomy is aspiration thrombectomy, laser thrombectomy or mechanical thrombectomy (e.g. rheolytic or rotating cutter)). Optionally, the method includes step d) inserting a stent, or conducting rotational or laser atherectomy, and/or brachytherapy. Optionally, a thrombolytic agent such as tPA is administered after determination that the patient has a myocardial infarction and prior to step c). The method can be used on a STEMI or NSTEMI MI.

In a further embodiment, the invention relates to a method of treating a patient having (no step b)), or suspected of having (include step b)), a pulmonary embolism comprising:
a) administering a diffusion enhancing compound to said patient within 4 (advantageously 2) hours of the first pulmonary embolism symptoms,
b) determining whether said patient has a pulmonary embolism, and if so determined,
c) performing a catheter based endovascular embolectomy on said patient.

Typically, the embolectomy is aspiration embolectomy, laser embolectomy or mechanical embolectomy. Optionally, a thrombolytic agent is also administered after determination that the patient has a pulmonary embolism and prior to step c). In another embodiment, step c) is replace with catheter directed thrombolysis.

The invention also relates to a method of treating a patient having (no step b)) or suspected of having (include step b)), a deep vein thrombosis comprising:
a) administering a diffusion enhancing compound to said patient as soon as possible, advantageously within 4 (more advantageously 2) hours of the first deep vein thrombosis symptoms,
b) determining whether said patient has a deep vein thrombosis, and if so determined, and
c) performing a catheter based endovascular thrombectomy on said patient.

Typically, the thrombectomy is aspiration thrombectomy or embolectomy, laser thrombectomy, or mechanical thrombectomy. Optionally, a thrombolytic agent is administered after determination that the patient has deep vein thrombosis. In another embodiment, step c) is replace with catheter directed thrombolysis.

In a further embodiment, the invention relates to a method of treating a patient having (no step b), or suspected of having (include step b)), a blood clot in a peripheral artery comprising:
a) administering a diffusion enhancing compound to said patient within 2 hours of the first blood clot in a peripheral artery symptoms,
b) determining whether said patient has a blood clot in a peripheral artery, and if so determined,
c) performing a catheter based endovascular thrombectomy or embolectomy on said patient.

Typically, the thrombectomy or embolectomy is aspiration thrombectomy or embolectomy, laser thrombectomy or embolectomy, or mechanical thrombectomy or embolectomy. Optionally, a thrombolytic agent is administered after step b). In another embodiment, step c) is replace with catheter directed thrombolysis.

In a still further embodiment, the invention relates to a method of reducing cell death in the penumbra of an embolism or thrombosis in a patient comprising:
a) administering a diffusion enhancing compound to said patient within 2 hours of the first symptom of the embolism or thrombosis, and
b) performing a catheter based endovascular thrombectomy or an embolectomy on said patient.

If the embolism or thrombosis is in the brain, the embolectomy is advantageously performed by a stent retriever or by aspiration. Optionally, a thrombolytic agent such as tPA is administered after step a) and before step b).

In any of the above methods, the diffusion enhancing compound is a bipolar trans carotenoid, advantageously a bipolar trans carotenoid salt (e.g. TSC). In a further embodiment, the trans carotenoid salt is formulated with a cyclodextrin. The diffusion enhancing compound is advantageously administered IV or IM. If the diffusion enhancing compound is TSC, a dose of about 0.05-2.5 mg/kg, advantageously a dose of about 0.2-2 mg/kg is used.

The thrombolytic agent utilized in the methods above is advantageously selected from the group consisting of tPA, reteplase, tenecteplase, anistreplase, streptokinase, and urokinase. The thrombolytic agent can be administered IV (infusion or bolus), or by catheter directed thrombolysis.

In all of the above methods, the diffusion enhancing compound is administered within 120, 90, 60, or most advantageously within 30 minutes of the onset of symptoms.

Although it is beneficial if the embolectomy or thrombectomy is performed as soon as possible after the embolism or thrombosis occurs (advantageously within 4 hours, more advantageously within 120, 90, or even 60 minutes), if the diffusion enhancing compound is administered within 4 hours of the onset of symptoms, the embolectomy or thrombectomy can be performed within 12 or even 24 hours of the onset of symptoms. In another embodiment, if the diffusion enhancing compound is administered within 3 hours of the onset of symptoms, the embolectomy or thrombectomy is performed within 9 hours of the onset of symptoms.

In a still further embodiment, the invention relates to a method of treating a patient suspected of having an embolism or thrombosis, or infarction, comprising administering a diffusion enhancing compound such as TSC by IV or IM injection to said patient within 60 minutes, 45 minutes or most advantageously 30 minutes of the first embolism or thrombosis symptoms.

The invention also relates to a kit comprising a first vial with a diffusion enhancing compound such as TSC (which can be lyophilized), a second vial with diluent such as water for injection, and a syringe for administration. The kit may be used for any of the methods described herein (e.g., any of the methods above or any of Methods 1-10 et seq. below).

The invention also includes a kit comprising:

a) a container comprising a diffusion enhancing compound such as TSC, and

b) instructions for using the diffusion enhancing compound to treat a patient having, or suspected of having, a thrombosis or embolism (e.g. ischemic stroke, heart attack, pulmonary embolism) or a hemorrhage (e.g. hemorrhagic stroke), by administering (advantageously within 90 minutes of the onset of symptoms) the diffusion enhancing compound at a dose of about 0.05-2.5 mg/kg to the patient. The kit may be used for any of the methods described herein (e.g., any of the methods above or any of Methods 1-10 et seq. below).

Further the invention relates to a double chamber container or syringe for separately holding in the two chambers (and combining just before administration): a) a solid, in particular a lyophilizate of a diffusion enhancing compound such as TSC, and b) a liquid reconstitution medium therefor such as water for injection. The container or syringe may be used in any of the methods described herein (e.g., any of the methods above or any of Methods 1-10 et seq. below).

### DETAILED DESCRIPTION OF THE INVENTION

The subject invention provides methods of rapid response to the treatment of patients suspected of having, or diagnosed as having an embolism or thrombosis. The invention relates to diffusion enhancing compounds and their use with embolectomy or thrombectomy for the treatment of disorders resulting from the formation of an embolus or thrombus—infarction—such as a myocardial infarction or stroke (brain infarction). Immediately following the event, blood flow and therefore oxygen transport is reduced locally, leading to hypoxia of the cells near the location of the original insult. Fast identification and treatment are crucial to limit cell death. The methods of the subject invention, via early (typically pre-hospital or emergency room) administration of a diffusion enhancing compound, and prompt embolectomy or thrombectomy, reduce cell death in the penumbra. The penumbra is an area surrounding an ischemic event of moderately ischemic tissue surrounding an area of more severe ischemic tissue. The methods of the subject invention enhance oxygen and glucose flow to this area to prevent the spread of the infarction.

The methods of the subject invention include administration of a therapeutically effective amount of a diffusion enhancing compound such as TSC within a short time (advantageously within 120 minutes, 90 minutes, 60 minutes, or even 30 minutes or less) of the onset of symptoms of an embolism or thrombosis (e.g. stroke symptoms, heart attack symptoms, pulmonary embolism symptoms, acute limb ischemia symptoms), typically by emergency medical personnel (e.g. paramedics), either upon arrival, or on the ambulance on the way to the hospital or at the emergency room (ER). The thrombectomy or embolectomy is then performed promptly at the hospital.

In one embodiment, provided is a method (Method 1, which includes Methods 1a and 1b below) of treating a patient (e.g., a human) having or suspected of having an embolism or thrombosis or infarction. For instance, provided is a method (Method 1a) of treating a patient (e.g., a human) having (omit step b below) or suspected of having an embolism or thrombosis or infarction comprising:
a) administering a diffusion enhancing compound to said patient (e.g., administering a diffusion enhancing compound to said patient within 6 hours (e.g., within 4 hours, advantageously within 2 hours) of the onset of symptoms of an embolism or thrombosis),
b) if suspected, determining whether said patient has an embolism or thrombosis, and if so determined,
c) performing an embolectomy or a thrombectomy (e.g., a catheter based endovascular embolectomy or thrombectomy or a surgical embolectomy or thrombectomy) on said patient.

For instance, provided is a method (Method 1b) of treating a patient (e.g., a human) having (omit step b below) or suspected of having an embolism or thrombosis or infarction comprising:
a) administering trans sodium crocetinate (TSC) to said patient (e.g., administering TSC to said patient within 6 hours (e.g., within 4 hours, advantageously within 2 hours) of the onset of symptoms of an embolism or thrombosis or infarction),
b) if suspected, determining whether said patient has an embolism or thrombosis or infarction, and if so determined,
c) performing an embolectomy or a thrombectomy (e.g., a catheter based endovascular embolectomy or thrombectomy or a surgical embolectomy or thrombectomy) on said patient.

Optionally, step c) in Method 1a or 1b is replaced with catheter directed thrombolysis.

In another embodiment, provided is a method (Method 2, which includes Methods 2a and 2b below) of treating a patient (e.g., a human) having or suspected of having an ischemic stroke. For instance, provided is a method (Method 2a) of treating a patient (e.g., a human) having (omit step b below) or suspected of having an ischemic stroke comprising:
a) administering a diffusion enhancing compound to said patient (e.g., administering a diffusion enhancing compound to said patient within 6 hours (e.g., within 4 hours, advantageously within 2 hours) of the onset of symptoms of the stroke),
b) if suspected, determining whether said patient has an ischemic stroke, and if so determined,
c) performing an embolectomy or a thrombectomy (e.g., a mechanical embolectomy or thrombectomy, e.g., a catheter based endovascular embolectomy or thrombectomy, e.g. using a stent retriever or aspiration device) on said patient.

For instance, provided is a method (Method 2b) of treating a patient (e.g., a human) having (omit step b below) or suspected of having an ischemic stroke comprising:
a) administering trans sodium crocetinate (TSC) to said patient (e.g., administering TSC to said patient within 6 hours (e.g., within 4 hours, advantageously within 2 hours) of the onset of symptoms of the stroke),
b) if suspected, determining whether said patient has an ischemic stroke, and if so determined,
c) performing an embolectomy or a thrombectomy (e.g., a mechanical embolectomy or thrombectomy, e.g., a catheter based endovascular embolectomy or thrombectomy, e.g. using a stent retriever or aspiration device) on said patient.

Optionally, in Method 2a or 2b, the embolectomy or thrombectomy is performed with: clot retrievers including stent retrievers or devices with a balloon that can pull out a clot; aspiration/suction devices including rheolytic devices; ultrasound based devices; laser based devices; or snare-like devices. Optionally, step c) in Method 2a or 2b is replaced with catheter directed thrombolysis.

In another embodiment, provided is a method (Method 3, which includes Methods 3a and 3b below) of treating a patient (e.g., a human) having a stroke where it is unknown whether the stroke is an ischemic stroke or a hemorrhagic stroke. For instance, provided is a method (Method 3a) of treating a patient (e.g., a human) having a stroke where it is unknown whether the stroke is an ischemic stroke or a hemorrhagic stroke comprising:
a) administering a diffusion enhancing compound to said patient (e.g., administering a diffusion enhancing compound to said patient within 6 hours (e.g., within 4 hours, advantageously within 2 hours) of the onset of symptoms of the stroke),
b) determining whether the stroke is an ischemic stroke, and if so determined,
c) performing an embolectomy or a thrombectomy (e.g., a mechanical embolectomy or thrombectomy, e.g., a catheter based endovascular embolectomy or thrombectomy, e.g. using a stent retriever or aspiration device) on said patient.

For instance, provided is a method (Method 3b) of treating a patient (e.g., a human) having a stroke where it is unknown whether the stroke is an ischemic stroke or a hemorrhagic stroke comprising:
a) administering trans sodium crocetinate (TSC) to said patient (e.g., administering TSC to said patient within 6 hours (e.g., within 4 hours, advantageously within 2 hours) of the onset of symptoms of the stroke),
b) determining whether the stroke is an ischemic stroke, and if so determined,
c) performing an embolectomy or a thrombectomy (e.g., a mechanical embolectomy or thrombectomy, e.g., a catheter based endovascular embolectomy or thrombectomy, e.g. using a stent retriever or aspiration device) on said patient.

Optionally, in Method 3a or 3b, the embolectomy or thrombectomy is performed with: clot retrievers including stent retrievers or devices with a balloon that can pull out a clot; aspiration/suction devices including rheolytic devices; ultrasound based devices; laser based devices; or snare-like devices. Optionally, step c) in Method 3a or 3b is replaced with catheter directed thrombolysis.

In another embodiment, provided is a method (Method 4, which includes Methods 4a and 4b below) of treating a patient (e.g., a human) having or suspected of having a myocardial infarction (e.g., STEMI or NSTEMI MI). For instance, provided is a method (Method 4a) of treating a patient (e.g., a human) having (omit step b below) or suspected of having a myocardial infarction (e.g., STEMI or NSTEMI MI) comprising:
a) administering a diffusion enhancing compound to said patient (e.g., administering a diffusion enhancing compound to said patient within 6 hours (e.g., within 4 hours, advantageously within 2 hours) of the onset of symptoms of a myocardial infarction),
b) if suspected, determining whether said patient has a myocardial infarction, and if so determined,
c) performing an embolectomy or a thrombectomy (e.g., a catheter based endovascular embolectomy or thrombectomy, e.g., aspiration thrombectomy or embolectomy, laser thrombectomy or embolectomy, or mechanical thrombectomy or embolectomy) on said patient.

For instance, provided is a method (Method 4b) of treating a patient (e.g., a human) having (omit step b below) or suspected of having a myocardial infarction (e.g., STEMI or NSTEMI MI) comprising:
a) administering trans sodium crocetinate (TSC) to said patient (e.g., TSC to said patient within 6 hours (e.g., within 4 hours, advantageously within 2 hours) of the onset of symptoms of a myocardial infarction),
b) if suspected, determining whether said patient has a myocardial infarction, and if so determined,
c) performing an embolectomy or a thrombectomy (e.g., a catheter based endovascular embolectomy or thrombectomy, e.g., aspiration thrombectomy or embolectomy, laser thrombectomy or embolectomy, or mechanical thrombectomy or embolectomy) on said patient.

Optionally, in Method 4a or 4b the thrombectomy or embolectomy is an aspiration thrombectomy or embolectom, laser thrombectomy or embolectomy, or mechanical thrombectomy or embolectomy (e.g., rheolytic or rotating cutter) and/or Method 4a or 4b includes step d) inserting a stent, or conducting rotational or laser atherectomy and/or brachytherapy.

In another embodiment, provided is a method (Method 5, which includes Methods 5a and 5b below) of treating a patient (e.g., a human) having or suspected of having a pulmonary embolism. For instance, provided is a method (Method 5a) of treating a patient (e.g., a human) having (omit step b below) or suspected of having a pulmonary embolism comprising:
a) administering a diffusion enhancing compound to said patient (e.g., administering a diffusion enhancing compound to said patient within 6 hours (e.g., within 4 hours, advantageously within 2 hours) of the onset of pulmonary embolism symptoms),
b) if suspected, determining whether said patient has a pulmonary embolism, and if so determined,
c) performing an embolectomy (e.g., a catheter based endovascular embolectomy, e.g., aspiration embolectomy, laser embolectomy, or mechanical embolectomy) on said patient.

For instance, provided is a method (Method 5b) of treating a patient (e.g., a human) having (omit step b below) or suspected of having a pulmonary embolism comprising:
a) administering trans sodium crocetinate (TSC) to said patient (e.g., administering TSC to said patient within 6 hours (e.g., within 4 hours, advantageously within 2 hours) of the onset of pulmonary embolism symptoms),
b) if suspected, determining whether said patient has a pulmonary embolism, and if so determined,
c) performing an embolectomy (e.g., a catheter based endovascular embolectomy, e.g., aspiration embolectomy, laser embolectomy or mechanical embolectomy) on said patient.

Optionally, step c) in Method 5a or 5b is replaced with catheter directed thrombolysis.

In another embodiment, provided is a method (Method 6 which includes Methods 6a and 6b below) of treating a patient (e.g., a human) having or suspected of having a deep vein thrombosis. For instance, provided is a method (Method 6a) of treating a patient (e.g., a human) having (omit step b below) or suspected of having a deep vein thrombosis comprising:
a) administering a diffusion enhancing compound to said patient (e.g., administering a diffusion enhancing compound to said patient within 6 hours (e.g., within 4 hours, advantageously within 2 hours) of the onset of deep vein thrombosis symptoms),
b) if suspected, determining whether said patient has a deep vein thrombosis, and if so determined,
c) performing an embolectomy or a thrombectomy (e.g., a catheter based endovascular thrombectomy or embolectomy, e.g., aspiration thrombectomy or embolectomy, laser thrombectomy or embolectomy, or mechanical thrombectomy or embolectomy) on said patient.

For instance, provided is a method (Method 6b) of treating a patient (e.g., a human) having (omit step b below) or suspected of having a deep vein thrombosis comprising:
a) administering trans sodium crocetinate (TSC) to said patient (e.g., administering TSC to said patient within 6 hours (e.g., within 4 hours, advantageously within 2 hours) of the onset of deep vein thrombosis symptoms),
b) if suspected, determining whether said patient has a deep vein thrombosis, and if so determined,
c) performing an embolectomy or a thrombectomy (e.g., a catheter based endovascular thrombectomy or embolectomy, e.g., aspiration thrombectomy or embolectomy, laser thrombectomy or embolectomy, or mechanical thrombectomy or embolectomy) on said patient.

Optionally, step c) in Method 6a or 6b is replaced with catheter directed thrombolysis.

In another embodiment, provided is a method (Method 7, which includes Methods 7a and 7b below) of treating a patient (e.g., a human) having or suspected of having a blood clot in a peripheral artery, acute limb ischemia, or mesenteric ischemia. For instance, provided is a method (Method 7a) of treating a patient (e.g., a human) having (omit step b below) or suspected of having a blood clot in a peripheral artery, acute limb ischemia, or mesenteric ischemia comprising:
a) administering a diffusion enhancing compound to said patient (e.g., administering a diffusion enhancing compound to said patient within 6 hours (e.g., within 4 hours, advantageously within 2 hours) of the onset of blood clot in a peripheral artery, acute limb ischemia, or mesenteric ischemia symptoms),
b) if suspected, determining whether said patient has a blood clot in a peripheral artery, acute limb ischemia, or mesenteric ischemia, and if so determined,
c) performing an embolectomy or a thrombectomy (e.g., a catheter based endovascular thrombectomy or embolectomy, e.g., aspiration thrombectomy or embolectomy, laser thrombectomy or embolectomy, or mechanical thrombectomy or embolectomy) on said patient.

For instance, provided is a method (Method 7b) of treating a patient (e.g., a human) having (omit step b below) or suspected of having a blood clot in a peripheral artery, acute limb ischemia, or mesenteric ischemia comprising:
a) administering trans sodium crocetinate (TSC) to said patient (e.g., administering TSC to said patient within 6 hours (e.g., within 4 hours, advantageously within 2 hours) of the onset of blood clot in a peripheral artery symptoms),
b) if suspected, determining whether said patient has a blood clot in a peripheral artery, acute limb ischemia, or mesenteric ischemia, and if so determined,
c) performing an embolectomy or a thrombectomy (e.g., a catheter based endovascular thrombectomy or embolectomy, e.g., aspiration thrombectomy or embolectomy, laser thrombectomy or embolectomy, or mechanical thrombectomy or embolectomy) on said patient.

Optionally, step c) in Method 7a or 7b is replaced with catheter directed thrombolysis.

In another embodiment, provided is a method (Method 8, which includes Methods 8a and 8b below) of reducing cell death in the penumbra of an embolism or thrombosis in a patient (e.g., a human) comprising. For instance, provided is a method (Method 8a) of reducing cell death in the penumbra of an embolism or thrombosis in a patient (e.g., a human) comprising:
a) administering a diffusion enhancing compound to said patient (e.g., administering a diffusion enhancing compound to said patient within 6 hours (e.g., within 4 hours, advantageously within 2 hours) of the first symptom of the embolism or thrombosis), and
b) performing a thrombectomy or an embolectomy (e.g., a catheter based endovascular thrombectomy or an embolectomy, e.g., if the embolism or thrombosis is in the brain, the embolectomy is advantageously performed by a stent retriever or by aspiration) on said patient.

For instance, provided is a method (Method 8b) of reducing cell death in the penumbra of an embolism or thrombosis in a patient (e.g., a human) comprising:
a) administering trans sodium crocetinate to said patient (e.g., administering TSC to said patient within 6 hours (e.g., within 4 hours, advantageously within 2 hours) of the first symptom of the embolism or thrombosis), and
b) performing a thrombectomy or an embolectomy (e.g., a catheter based endovascular thrombectomy or an embolectomy, e.g., if the embolism or thrombosis is in the brain, the embolectomy is advantageously performed by a stent retriever or by aspiration) on said patient.

In another embodiment, provided is a method (Method 9, which includes Methods 9a and 9b below) of treating a patient (e.g., a human) having or suspected of having a myocardial infarction (MI). For instance, provided is a method (Method 9a) of treating a patient (e.g., a human) having (omit step b below) or suspected of having a myocardial infarction (MI) comprising:
a) administering a diffusion enhancing compound to said patient (e.g., administering a diffusion enhancing compound to said patient within 6 hours (e.g., within 4 hours, advantageously within 2 hours) of the onset of MI symptoms),
b) if suspected, determining whether said patient has a myocardial infarction, and if so determined,
c) performing percutaneous coronary intervention (PCI) on said patient.

For instance, provided is a method (Method 9a) of treating a patient (e.g., a human) having (omit step b below) or suspected of having a myocardial infarction (MI) comprising:
a) administering trans sodium crocetinate (TSC) to said patient (e.g., administering TSC to said patient within 6 hours (e.g., within 4 hours, advantageously within 2 hours) of the onset of MI symptoms),
b) if suspected, determining whether said patient has a myocardial infarction, and if so determined,
c) performing percutaneous coronary intervention (PCI) on said patient.

Optionally, in Method 9a or 9b the clot causing the MI is removed (e.g. using aspiration or laser) prior to performing PCI and/or step c) in Method 9a or 9b includes rotational or laser atherectomy and/or brachytherapy. Optionally, step c) in Method 9a or 9b is replaced with catheter directed thrombolysis.

In another embodiment, provided is a method (Method 10, which includes Methods 10a and 10b below) of treating a patient (e.g. a human) having a hemorrhagic stroke. For instance, provided is a method (Method 10a) of treating a patient (e.g. a human) having a hemorrhagic stroke comprising:
a) administering a diffusion enhancing compound to said patient (e.g., administering a diffusion enhancing compound to said patient within 6 hours (e.g., within 4 hours, advantageously within 2 hours) of the first stroke symptoms), and
b) inserting a coil or clipping the artery at the site of the hemorrhage in said patient.

For instance, provided is a method (Method 10b) of treating a patient (e.g. a human) having a hemorrhagic stroke comprising:
a) administering trans sodium crocetinate (TSC) to said patient (e.g., administering a diffusion enhancing compound to said patient within 6 hours (e.g., within 4 hours, advantageously within 2 hours) of the first stroke symptoms), and
b) inserting a coil or clipping the artery at the site of the hemorrhage in said patient.

Further provided are any one of Methods 1-10 as follows:
1.1. Method 1a, wherein the diffusion enhancing compound is administered to said patient within 6 hours (e.g., within 4 hours, advantageously within 2 hours) of the onset of symptoms of an embolism or thrombosis or within 6 hours (e.g., within 4 hours, advantageously within 2 hours) of diagnosis of the suspected embolism or thrombosis or diagnosis of the embolism or thrombosis. Method 1b, wherein TSC is administered to said patient within 6 hours (e.g., within 4 hours, advantageously within 2 hours) of the onset of symptoms of an embolism or thrombosis or within 6 hours (e.g., within 4 hours, advantageously within 2 hours) of diagnosis of the suspected embolism or thrombosis or diagnosis of the embolism or thrombosis.
1.2. Method 2a, wherein the diffusion enhancing compound is administered to said patient within 6 hours (e.g., within 4 hours, advantageously within 2 hours) of the onset of symptoms of an ischemic stroke or within 6 hours (e.g., within 4 hours, advantageously within 2 hours) of diagnosis of the suspected ischemic stroke or diagnosis of the ischemic stroke. Method 2b, wherein TSC is administered to said patient within 6 hours (e.g., within 4 hours, advantageously within 2 hours) of the onset of symptoms of an ischemic stroke or within 6 hours (e.g., within 4 hours, advantageously within 2 hours) of diagnosis of the suspected ischemic stroke or diagnosis of the ischemic stroke. Method 3a, wherein the diffusion enhancing compound is administered to said patient within 6 hours (e.g., within 4 hours, advantageously within 2 hours) of the onset of symptoms of a stroke or within 6 hours (e.g., within 4 hours, advantageously within 2 hours) of diagnosis of the stroke. Method 3b, wherein TSC is administered to said patient within 6 hours (e.g., within 4 hours, advantageously within 2 hours) of the onset of symptoms of a stroke or within 6 hours (e.g., within 4 hours, advantageously within 2 hours) of diagnosis of the stroke.
1.3. Method 4a, wherein the diffusion enhancing compound is administered to said patient within 6 hours (e.g., within 4 hours, advantageously within 2 hours) of the onset of symptoms of a myocardial infarction or within 6 hours (e.g., within 4 hours, advantageously within 2 hours) of diagnosis of the suspected myocardial infarction or diagnosis of the myocardial infarction. Method 4b, wherein TSC is administered to said patient within 6 hours (e.g., within 4 hours, advantageously within 2 hours) of the onset of symptoms of a myocardial infarction or within 6 hours (e.g., within 4 hours, advantageously within 2 hours) of diagnosis of the suspected myocardial infarction or diagnosis of the myocardial infarction.
1.4. Method 5a, wherein the diffusion enhancing compound is administered to said patient within 6 hours (e.g., within 4 hours, advantageously within 2 hours) of the onset of pulmonary embolism symptoms or within 6 hours (e.g., within 4 hours, advantageously within 2 hours) of diagnosis of the suspected pulmonary embolism or diagnosis of the pulmonary embolism. Method 5b, wherein TSC is administered to said patient within 6 hours (e.g., within 4 hours, advantageously within 2 hours) of the onset of pulmonary embolism symptoms or within 6 hours (e.g., within 4 hours, advantageously within 2 hours) of diagnosis of the suspected pulmonary embolism or diagnosis of the pulmonary embolism.
1.5. Method 6a, wherein the diffusion enhancing compound is administered to said patient within 6 hours (e.g., within 4 hours, advantageously within 2 hours) of the onset of deep vein thrombosis symptoms or within 6 hours (e.g., within 4 hours, advantageously within 2 hours) of diagnosis of the suspected deep vein thrombosis or diagnosis of the deep vein thrombosis. Method 6b, wherein TSC is administered to said patient within 6 hours (e.g., within 4 hours, advantageously within 2 hours) of the onset of deep vein thrombosis symptoms or within 6 hours (e.g., within 4 hours, advantageously within 2 hours) of diagnosis of the suspected deep vein thrombosis or diagnosis of the deep vein thrombosis.
1.6. Method 7a, wherein the diffusion enhancing compound is administered to said patient within 6 hours (e.g., within 4 hours, advantageously within 2 hours) of the onset of a blood clot in a peripheral artery, acute limb ischemia, or mesenteric ischemia symptoms or within 6 hours (e.g., within 4 hours, advantageously within 2 hours) of diagnosis of the suspected blood clot in a peripheral artery, acute limb ischemia, or mesenteric ischemia or diagnosis of the blood clot in a peripheral artery, acute limb ischemia, or mesenteric ischemia. Method 7b, wherein TSC is administered to said patient within 6 hours (e.g., within 4 hours, advantageously within 2 hours) of the onset of a blood clot in a peripheral artery symptoms or within 6 hours (e.g., within 4 hours, advantageously within 2 hours) of the diagnosis of the suspected blood clot in a peripheral artery, acute limb ischemia, or mesenteric ischemia, or diagnosis of the blood clot in a peripheral artery, acute limb ischemia, or mesenteric ischemia.
1.7. Method 8a, wherein the diffusion enhancing compound is administered to said patient within 6 hours (e.g., within 4 hours, advantageously within 2 hours) of the first symptom of an embolism or a thrombosis or within 6 hours (e.g., within 4 hours, advantageously within 2 hours) of the diagnosis of the embolism or thrombosis. Method 8b, wherein TSC is administered to said patient within 6 hours (e.g., within 4 hours, advantageously within 2 hours) of the first symptom of an embolism or a thrombosis or within 6 hours (e.g., within 4 hours, advantageously within 2 hours) of the diagnosis of the embolism or thrombosis.
1.8. Method 9a, wherein the diffusion enhancing compound is administered to said patient within 6 hours (e.g., within 4 hours, advantageously within 2 hours) of the onset of MI symptoms or within 6 hours (e.g., within 4 hours, advantageously within 2 hours) of the diagnosis of the suspected MI of diagnosis of the MI. Method 9b, wherein TSC is administered to said patient within 6 hours (e.g., within 4 hours, advantageously within 2 hours) of the onset of MI symptoms or within 6 hours (e.g., within 4 hours, advantageously within 2 hours) of the diagnosis of the suspected MI or diagnosis of the MI.
1.9. Method 10a, wherein the diffusion enhancing compound is administered to said patient within 6 hours (e.g., within 4 hours, advantageously within 2 hours) of the first stroke symptoms or within 6 hours (e.g., within 4 hours, advantageously within 2 hours) of diagnosis of the hemorrhagic stroke. Method 10b, wherein TSC is administered to said patient within 6 hours (e.g., within 4 hours, advantageously within 2 hours) of the first stroke symptoms or within 6 hours (e.g., within 4 hours, advantageously within 2 hours) of diagnosis of the hemorrhagic stroke.
1.10. Any one of Methods 1a, 1b, 2a, 2b, 3a, 3b, 4a, 4b, 5a, 5b, 6a, 6b, 7a, 7b, 8a, 8b, 9a, 9b, or 1.1-1.8, wherein said embolectomy or thrombectomy is any discussed herein, e.g., a catheter based embolectomy or thrombectomy or a surgical embolectomy or thrombectomy. Any one of Methods 1a, 1b, 2a, 2b, 3a, 3b, 4a, 4b, 5a, 5b, 6a, 6b, 7a, 7b, 8a, 8b, 9a, 9b, or 1.1-1.8, wherein said embolectomy or thrombectomy is a mechanical embolectomy or thrombectomy. Any one of Methods 1a, 1b, 2a, 2b, 3a, 3b, 4a, 4b, 5a, 5b, 6a, 6b, 7a, 7b, 8a, 8b, 9a, 9b, or 1.1-1.8, wherein said embolectomy or thrombectomy is an aspiration embolectomy or thrombectomy or laser embolectomy or thrombectomy.
1.11. Any one of Methods 1a, 1b, 2a, 2b, 3a, 3b, 4a, 4b, 5a, 5b, 6a, 6b, 7a, 7b, 8a, 8b, 9a, 9b, 1.1-1.8, or 1.10, wherein said embolectomy or thrombectomy is performed with a stent retriever. Any one of Methods 1a, 1b, 2a, 2b, 3a, 3b, 4a, 4b, 5a, 5b, 6a, 6b, 7a, 7b, 8a, 8b, 9a, 9b, 1.1-1.8, or 1.10, wherein said embolectomy or thrombectomy is performed with an aspiration device.
1.12. Any one of Methods 4a, 4b, or 1.3 further comprising performing a thrombolectomy prior to step c).
1.13. Any one of Methods 4a, 4b, 1.3, or 1.12, wherein said thrombectomy is aspiration thrombectomy.
1.14. Any one of Methods 4a, 4b, 1.3, 1.12, or 1.13, wherein the myocardial infarction is STEMI.
1.15. Any one of Methods 8a, 8b, or 1.7, wherein the penumbra is in the brain or heart.
1.16. Any one of Methods 9a, 9b, or 1.8, wherein the clot is removed prior to performing PCI.
1.17. Any one of Methods 9a, 9b, 1.8, or 1.16, wherein the PCI includes rotational or laser atherectomy and/or brachytherapy.
1.18. Any one of Methods 9a, 9b, 1.8, 1.16, or 1.17 further comprising performing an embolectomy or a thrombectomy prior to step c). For instance, any one of Methods 9a, 9b, 1.8, 1.16, or 1.17 further comprising performing a thrombectomy prior to step c).
1.19. Method 1.18, wherein said thrombectomy is aspiration thrombectomy.
1.20. Any one of Methods 1a, 1b, 2a, 2b, 3a, 3b, 4a, 4b, 5a, 5b, 6a, 6b, 7a, 7b, 8a, 8b, 1.1-1.7, or 1.10-1.15, wherein said embolectomy or thrombectomy is a catheter based endovascular embolectomy or thrombectomy.
1.21. Any one of Methods 1a, 1b, 2a, 2b, 3a, 3b, 4a, 4b, 5a, 5b, 6a, 6b, 7a, 7b, 8a, 8b, 1.1-1.7, or 1.10-1.15, wherein said embolectomy or thrombectomy is a surgical embolectomy or thrombectomy.
1.22. Any one of Methods 1a, 1b, 2a, 2b, 3a, 3b, 4a, 4b, 5a, 5b, 6a, 6b, 7a, 7b, 8a, 8b, 1.1-1.7, or 1.10-1.15, wherein said embolectomy or thrombectomy is performed by a balloon device or aspiration device.
1.23. Any one of Methods 1a, 1b, 2a, 2b, 3a, 3b, 4a, 4b, 5a, 5b, 6a, 6b, 7a, 7b, 8a, 8b, 9a, 9b, 10a, 10b, or 1.1-1.22, wherein said administration in step a) is within 90 minutes (e.g., within 60 minutes) of the onset of symptoms or diagnosis.
1.24. Any one of Methods 1a, 2a, 3a, 4a, 5a, 6a, 7a, 8a, 9a, 10a, or 1.1-1.23, wherein said diffusion enhancing compound is a bipolar trans carotenoid salt.
1.25. Any one of Methods 1a, 2a, 3a, 4a, 5a, 6a, 7a, 8a, 9a, 10a, or 1.1-1.24, wherein said diffusion enhancing compound is a bipolar trans carotenoid salt having the formula:

YZ-TCRO-ZY,
where:
Y=a cation which can be the same or different,
Z=a polar group which can the same or different and which is associated with the cation,
TCRO=a linear trans carotenoid skeleton with conjugated carbon-carbon double bonds and single bonds, and having pendant groups X, wherein the pendant groups X, which can be the same or different, are a linear or branched hydrocarbon group having 10 or less carbon atoms, or a halogen.
1.26. Method 1.24 or 1.25, wherein the bipolar trans carotenoid salt is trans sodium crocetinate (TSC) (e.g., synthetic TSC).
1.27. Any one of Methods 1.24-1.26, wherein the absorbency (e.g., in an aqueous solution) of the bipolar trans carotenoid salt (e.g., trans sodium crocetinate) at the highest peak which occurs in the visible wavelength range divided by the absorbency of a peak occurring in the ultraviolet wavelength range is greater than 7 (e.g., 7 to 8.5), e.g., greater than 7.5 (e.g., 7.5 to 9, e.g., 7.5 to 8.5), e.g., greater than 8 (e.g., 8 to 8.8), e.g., greater than 8.5. Any one of Methods 1b, 2b, 3b, 4b, 5b, 6b, 7b, 8b, 9b, 10b, or 1.1-1.24, wherein the absorbency (e.g., in an aqueous solution) of the TSC at the highest peak which occurs in the visible wavelength range divided by the absorbency of a peak occurring in the ultraviolet wavelength range is greater than 7 (e.g., 7 to 8.5), e.g., greater than 7.5 (e.g., 7.5 to 9, e.g., 7.5 to 8.5), e.g., greater than 8 (e.g., 8 to 8.8), e.g., greater than 8.5.
1.28. Any one of Methods 1.24-1.27, wherein the bipolar trans carotenoid salt (e.g., trans sodium crocetinate) is at least 90% pure as measured by high performance liquid chromatography (HPLC), e.g., ≥95% pure as measured by HPLC, e.g., ≥96% pure as measured by HPLC. Any one of Methods 1b, 2b, 3b, 4b, 5b, 6b, 7b, 8b, 9b, 10b, 1.1-1.24, or 1.27, wherein the TSC is at least 90% pure as measured by high performance liquid chromatography (HPLC), e.g., ≥95% pure as measured by HPLC, e.g., ≥96% pure as measured by HPLC.
1.29. Any one of Methods 1.24-1.28, wherein the bipolar trans carotenoid salt is in a composition also comprising a cyclodextrin. For instance, wherein TSC is in a composition also comprising a cyclodextrin (e.g., wherein the TSC is in a lyophilized composition with a cyclodextrin).
1.30. Method 1.29, wherein the cyclodextrin is gamma-cyclodextrin. For instance, wherein the bipolar trans carotenoid salt is TSC which is in a composition also comprising gamma-cyclodextrin (e.g., wherein the TSC is in a lyophilized composition with gamma-cyclodextrin).
1.31. Method 1.29 or 1.30, wherein the composition further comprises mannitol.
1.32. Any one of Methods 1a, 2a, 3a, 4a, 5a, 6a, 7a, 8a, 9a, 10a, or 1.1-1.31, wherein the diffusion enhancing compound is administered intravenously or intramuscularly (e.g., as an intravenous injection or infusion or intramuscular injection). For instance, any one of Methods 1a, 2a, 3a, 4a, 5a, 6a, 7a, 8a, 9a, 10a, or 1.1-1.31, wherein the diffusion enhancing compound is admixed with sterile water for injection to form an injection. Any one of Methods 1b, 2b, 3b, 4b, 5b, 6b, 7b, 8b, 9b, 10b, or 1.1-1.31, wherein TSC is administered intravenously or intramuscularly (e.g., as an intravenous injection or infusion or intramuscular injection). For instance, any one of Methods 1b, 2b, 3b, 4b, 5b, 6b, 7b, 8b, 9b, 10b, or 1.1-1.31, wherein TSC is admixed with sterile water for injection to form an injection.
1.33. Any one of Methods 1a, 2a, 3a, 4a, 5a, 6a, 7a, 8a, 9a, 10a, or 1.1.1-32, wherein the diffusion enhancing compound is TSC and is administered at a dose of 0.05-2.5 mg/kg, e.g., 0.2-2 mg/kg, e.g., 0.15-0.35 mg/kg, e.g., 0.25 mg/kg. Any one of Methods 1b, 2b, 3b, 4b, 5b, 6b, 7b, 8b, 9b, 10b, or 1.1.1-32, wherein TSC is administered at a dose of 0.05-2.5 mg/kg, e.g., 0.2-2 mg/kg, e.g., 0.15-0.35 mg/kg, e.g., 0.25 mg/kg.
1.34. Any one of Methods 1a, 1b, 2a, 2b, 3a, 3b, 4a, 4b, 5a, 5b, 6a, 6b, 7a, 7b, 9a, 9b, 1.1-1.6, 1.8, 1.10-1.14, or 1.16-1.33, wherein the method further comprises the administration of a thrombolytic agent prior to step c) (for stroke, the method must determine that the stroke is an ischemic stroke prior to administration of the thrombolytic agent). Any one of Methods 8a, 8b, 1.7, 1.10, 1.11, 1.15, or 1.20-1.33, wherein a thrombolytic agent such as tPA is administered after step a) and before step b).
1.35. Method 1.34, wherein said thrombolytic agent is selected from the group consisting of tPA, reteplase, tenecteplase, anistreplase, streptokinase, and urokinase.
1.36. Any one of Methods 1a, 2a, 3a, 4a, 5a, 6a, 7a, 8a, 9a, 10a, or 1.1-1.35, wherein the diffusion enhancing compound is administered within 120 minutes (e.g., within 90 minutes, e.g., within 60 minutes, e.g., advantageously within 30 minutes) of the onset of symptoms or diagnosis of the suspected embolism, suspected thrombosis, suspected ischemic stroke, ischemic stroke, stroke, suspected myocardial infarction, myocardial infarction, suspected pulmonary embolism, pulmonary embolism, suspected deep vein thrombosis, deep vein thrombosis, suspected blood clot in a peripheral artery, suspected acute limb ischemia, suspected mesenteric ischemia, blood clot in a peripheral artery, acute limb ischemia, mesenteric ischemia, embolism, thrombosis, or hemorrhagic stroke. Any one of Methods 1b, 2b, 3b, 4b, 5b, 6b, 7b, 8b, 9b, 10b, or 1.1-1.35, wherein TSC is administered within 120 minutes (e.g., within 90 minutes, e.g., within 60 minutes, e.g., advantageously within 30 minutes) of the onset of symptoms or diagnosis of the suspected embolism, suspected thrombosis, suspected ischemic stroke, ischemic stroke, stroke, suspected myocardial infarction, myocardial infarction, suspected pulmonary embolism, pulmonary embolism, suspected deep vein thrombosis, deep vein thrombosis, suspected blood clot in a peripheral artery, suspected acute limb ischemia, suspected mesenteric ischemia, blood clot in a peripheral artery, acute limb ischemia, mesenteric ischemia, embolism, thrombosis, or hemorrhagic stroke.
1.37. Any one of Methods 1a, 2a, 3a, 4a, 5a, 6a, 7a, 8a, 9a, 10a, or 1.1-1.36, wherein the diffusion enhancing compound is administered within 6 hours (e.g., within 4 hours, advantageously within 2 hours) and the embolectomy or thrombectomy is performed within 24 hours, e.g., within 16 hours, e.g., within 12 hours, e.g., within 9 hours, e.g., within 6 hours, of the onset of symptoms or diagnosis of the suspected embolism, suspected thrombosis, suspected ischemic stroke, ischemic stroke, stroke, suspected myocardial infarction, myocardial infarction, suspected pulmonary embolism, pulmonary embolism, suspected deep vein thrombosis, deep vein thrombosis, suspected blood clot in a peripheral artery, suspected acute limb ischemia, suspected mesenteric ischemia, blood clot in a peripheral artery, acute limb ischemia, mesenteric ischemia, embolism, thrombosis, or hemorrhagic stroke. Any one of Methods 1a, 2a, 3a, 4a, 5a, 6a, 7a, 8a, 9a, 10a, or 1.1-1.36, wherein the diffusion enhancing compound is administered within 3 hours (e.g., within 2 hours) and the embolectomy or thrombectomy is performed within 9 hours, e.g., within 6 hours, of the onset of symptoms or diagnosis of the suspected embolism, suspected thrombosis, suspected ischemic stroke, ischemic stroke, stroke, suspected myocardial infarction, myocardial infarction, suspected pulmonary embolism, pulmonary embolism, suspected deep vein thrombosis, deep vein thrombosis, suspected blood clot in a peripheral artery, suspected acute limb ischemia, suspected mesenteric ischemia, blood clot in a peripheral artery, acute limb ischemia, mesenteric ischemia, embolism, thrombosis, or hemorrhagic stroke. Any one of Methods 1b, 2b, 3b, 4b, 5b, 6b, 7b, 8b, 9b, 10b, or 1.1-1.36, wherein TSC is administered within 6 hours (e.g., within 4 hours, advantageously within 2 hours) and the embolectomy or thrombectomy is performed within 24 hours, e.g., within 16 hours, e.g., within 12 hours, e.g., within 9 hours, of the onset of symptoms or diagnosis of the suspected embolism, suspected thrombosis, suspected ischemic stroke, ischemic stroke, stroke, suspected myocardial infarction, myocardial infarction, suspected pulmonary embolism, pulmonary embolism, suspected deep vein thrombosis, deep vein thrombosis, suspected blood clot in a peripheral artery, suspected acute limb ischemia, suspected mesenteric ischemia, blood clot in a peripheral artery, acute limb ischemia, mesenteric ischemia, embolism, thrombosis, or hemorrhagic stroke. Any one of Methods 1b, 2b, 3b, 4b, 5b, 6b, 7b, 8b, 9b, 10b, or 1.1-1.36, wherein TSC is administered within 3 hours (e.g., within 2 hours) and the embolectomy or thrombectomy is performed within 9 hours, e.g., within 6 hours, of the onset of symptoms or diagnosis of the suspected embolism, suspected thrombosis, suspected ischemic stroke, ischemic stroke, stroke, suspected myocardial infarction, myocardial infarction, suspected pulmonary embolism, pulmonary embolism, suspected deep vein thrombosis, deep vein thrombosis, suspected blood clot in a peripheral artery, suspected acute limb ischemia, suspected mesenteric ischemia, blood clot in a peripheral artery, acute limb ischemia, mesenteric ischemia, embolism, thrombosis, or hemorrhagic stroke.

In another embodiment, provided is a diffusion enhancing compound (e.g., a bipolar trans carotenoid salt (e.g., TSC), e.g., as described in any one of Methods 1a, 1b, 2a, 2b, 3a, 3b, 4a, 4b, 5a, 5b, 6a, 6b, 7a, 7b, 8a, 8b, 9a, 9b, 10a, 10b, or 1.1-1.37) for use in any one of Methods 1a, 1b, 2a, 2b, 3a, 3b, 4a, 4b, 5a, 5b, 6a, 6b, 7a, 7b, 8a, 8b, 9a, 9b, 10a, 10b, or 1.1-1.37.

In another embodiment, provided is use of a diffusion enhancing compound (e.g., a bipolar trans carotenoid salt (e.g., TSC), e.g., as described in any one of Methods 1a, 1b, 2a, 2b, 3a, 3b, 4a, 4b, 5a, 5b, 6a, 6b, 7a, 7b, 8a, 8b, 9a, 9b, 10a, 10b, or 1.1-1.37) in the manufacture of a medicament for any one of Methods 1a, 1b, 2a, 2b, 3a, 3b, 4a, 4b, 5a, 5b, 6a, 6b, 7a, 7b, 8a, 8b, 9a, 9b, 10a, 10b, or 1.1-1.37.

In another embodiment, provided is a pharmaceutical composition comprising an effective amount of a diffusion enhancing compound (e.g., a bipolar trans carotenoid salt (e.g., TSC), e.g., as described in any one of Methods 1a, 1b, 2a, 2b, 3a, 3b, 4a, 4b, 5a, 5b, 6a, 6b, 7a, 7b, 8a, 8b, 9a, 9b, 10a, 10b, or 1.1-1.37) for use in any one of Methods 1a, 1b, 2a, 2b, 3a, 3b, 4a, 4b, 5a, 5b, 6a, 6b, 7a, 7b, 8a, 8b, 9a, 9b, 10a, 10b, or 1.1-1.37.

### Compounds and Compositions of the Invention

Diffusion Enhancing Compounds

The diffusion enhancing compounds of the invention include those compounds described in U.S. Pat. Nos. 7,759,506, 8,030,350, 8,901,174 and 8,206,751, each of which is hereby incorporated by reference in its entirety.

Included are bipolar trans carotenoid compounds having the formula:

YZ-TCRO-ZY

where:
Y=a cation
Z=a polar group which is associated with the cation, and
TCRO=trans carotenoid skeleton,
   such as TSC.

More specifically, the subject invention relates to trans carotenoids including trans carotenoid diesters, dialcohols, diketones and diacids, bipolar trans carotenoids (BTC), and bipolar trans carotenoid salts (BTCS) compounds and synthesis of such compounds having the structure:

YZ-TCRO-ZY

where:
Y (which can be the same or different at the two ends)=H or a cation other than H, preferably Na⁺ or K+ or Li⁺. Y is advantageously a monovalent metal ion. Y can also be an organic cation, e.g., R₄N⁺, R₃ S⁺, where R is H, or CₙH₂ₙ₊₁ where n is 1-10, advantageously 1-6. For example, R can be methyl, ethyl, propyl or butyl.
Z (which can be the same or different at the two ends)=polar group which is associated with H or the cation. Optionally including the terminal carbon on the carotenoid (or carotenoid related compound), this group can be a carboxyl (COO⁻) group or a CO group (e.g. ester, aldehyde or ketone group), or a hydroxyl group. This group can also be a sulfate group (OSO₃⁻) or a monophosphate group (OPO₃⁻), (OP(OH)O₂⁻), a diphosphate group, triphosphate or combinations thereof. This group can also be an ester group of COOR where the R is CₙH₂ₙ₋₁.
TCRO=trans carotenoid or carotenoid related skeleton (advantageously less than 100 carbons) which is linear, has pendant groups (defined below), and typically comprises “conjugated” or alternating carbon-carbon double and single bonds (in one embodiment, the TCRO is not fully conjugated as in a lycopene). The pendant groups (X) are typically methyl groups but can be other groups as discussed below. In an advantageous embodiment, the units of the skeleton are joined in such a manner that their arrangement is reversed at the center of the molecule. The 4 single bonds that surround a carbon-carbon double bond all lie in the same plane. If the pendant groups are on the same side of the carbon-carbon double bond, the groups are designated as cis (also known as “Z”); if they are on the opposite side of the carbon-carbon bond, they are designated as trans (also known as “E”). Throughout this case, the isomers will be referred to as cis and trans.

The compounds of the subject invention are trans. The cis isomer typically is a detriment—and results in the diffusivity not being increased. In one embodiment, a cis isomer can be utilized where the skeleton remains linear. The placement of the pendant groups can be symmetric relative to the central point of the molecule or can be asymmetric so that the left side of the molecule does not look the same as the right side of the molecule either in terms of the type of pendant group or their spatial relationship with respect to the center carbon.

The pendant groups X (which can be the same or different) are hydrogen (H) atoms, or a linear or branched hydrocarbon group having 10 or less carbons, advantageously 4 or less, (optionally containing a halogen), or a halogen. X could also be an ester group (COO—) or an ethoxy/methoxy group. Examples of X are a methyl group (CH₃), an ethyl group (C₂H₅), a phenyl or single aromatic ring structure with or without pendant groups from the ring, a halogen-containing alkyl group (C1-C10) such as CH₂Cl, or a halogen such as Cl or Br or a methoxy (OCH₃) or ethoxy (OCH₂CH₃). The pendant groups can be the same or different but the pendant groups utilized must maintain the skeleton as linear.

Although many carotenoids exist in nature, carotenoid salts do not. Commonly-owned U.S. Pat. No. 6,060,511 hereby incorporated by reference in its entirety, relates to trans sodium crocetinate (TSC). The TSC was made by reacting naturally occurring saffron with sodium hydroxide followed by extractions that selected primarily for the trans isomer.

The presence of the cis and trans isomers of a carotenoid or carotenoid salt can be determined by looking at the ultraviolet-visible spectrum for the carotenoid sample dissolved in an aqueous solution. Given the spectrum, the value of the absorbence of the highest peak which occurs in the visible wave length range of 380 to 470 nm (the number depending on the solvent used and the chain length of the BTC or BTCS. The addition of pendant groups or differing chain lengths will change this peak absorbance but someone skilled in the art will recognize the existence of an absorbance peak in the visible range corresponding to the conjugated backbone structure of these molecules.) is divided by the absorbency of the peak which occurs in the UV wave length range of 220 to 300 nm can be used to determine the purity level of the trans isomer. When the trans carotenoid diester (TCD) or BTCS is dissolved in water, the highest visible wave length range peak will be at between 380 nm to 470 nm (depending on the exact chemical structure, backbone length and pendant groups) and the UV wave length range peak will be between 220 to 300 nm. According to M. Craw and C. Lambert, Photochemistry and Photobiology, Vol. 38 (2), 241-243 (1983) hereby incorporated by reference in its entirety, the result of the calculation (in that case crocetin was analyzed) was 3.1, which increased to 6.6 after purification.

Performing the Craw and Lambert analysis, using a cuvette designed for UV and visible wavelength ranges, on the trans sodium salt of crocetin of commonly owned U.S. Pat. No. 6,060,511 (TSC made by reacting naturally occurring saffron with sodium hydroxide followed by extractions which selected primarily for the trans isomer), the value obtained averages about 6.8. Performing that test on the synthetic TSC of the subject invention, that ratio is greater than 7.0 (e.g. 7.0 to 8.5), advantageously greater than 7.5 (e.g. 7.5-8.5), most advantageously greater than 8. The synthesized material is a “purer” or highly purified trans isomer.

Thrombolytics

Thrombolysis is used in myocardial infarction (heart attack), ischemic strokes, deep vein thrombosis and pulmonary embolism to clear a blocked artery, i.e. a thrombus, and avoid permanent damage to the affected tissue (e.g. myocardium, brain, leg) and death. A less frequent use is to clear blocked catheters that are used in long-term medical therapy.

After it is determined that a thrombus is present, in addition to administering a bipolar trans carotenoid such as TSC, a therapeutically effective amount, i.e. a clot dissolving amount, of the thrombolytic agent such as tPA, can be administered.

The thrombolytic drugs include:
tissue plasminogen activator—t-PA—alteplase (Activase)
reteplase (Retavase)
tenecteplase (TNKase)
anistreplase (Eminase)
streptokinase (Kabikinase, Streptase)
urokinase (Abbokinase)

These drugs are most effective if administered immediately after it has been determined they are clinically appropriate.

Blood Thinners, Oxygen and Oxygen Carriers

The above drugs can be given in combination with intravenous heparin, or low molecular weight heparin, which are anticoagulant drugs. In a still further embodiment, oxygen and/or an artificial oxygen carrier (such as modified hemoglobin solutions or a perfluorochemical) is administered to elevate oxygen levels.

Formulation and Administration of the Compounds and Compositions of the Invention

A detailed description of formulation and administration of diffusing enhancing compounds can be found in commonly owned U.S. Pat. No. 8,293,804, U.S. application Ser. No. 12/801,726, and U.S. Pat. No. 8,206,751, each of which is hereby incorporated by reference in its entirety. A detailed description of formulation and administration of diffusing enhancing compounds can also be found in commonly owned U.S. Pat. No. 8,030,350, which is hereby incorporated by reference in its entirety.

A diffusion enhancing compound such as TSC can be administered by various routes for rapid delivery to the hypoxic tissue. For example, the compound, which can be formulated with other compounds including excipients, can be administered at the proper dosage as an intravenous injection (IV) or infusion, or an intramuscular injection (IM).

The IV injection route is an advantageous route for giving TSC for many of the uses of the subject application. Typically, a diffusion enhancing compound such as TSC is administered as soon as possible if a thrombus is believed present.

Cyclodextrins

In order to administer some pharmaceuticals, it is necessary to add another compound which will aid in increasing the absorption/solubility/concentration of the active pharmaceutical ingredient (API). Such compounds are called excipients, and cyclodextrins are examples of excipients. Cyclodextrins are cyclic carbohydrate chains derived from starch. They differ from one another by the number of glucopyranose units in their structure. The parent cyclodextrins contain six, seven and eight glucopyranose units, and are referred to as alpha, beta and gamma cyclodextrins respectively. Cyclodextrins were first discovered in 1891, and have been used as part of pharmaceutical preparations for several years.

Cyclodextrins are cyclic (alpha-1,4)-linked oligosaccharides of alpha-D-gluco-pyranose containing a relatively hydrophobic central cavity and hydrophilic outer surface. In the pharmaceutical industry, cyclodextrins have mainly been used as complexing agents to increase the aqueous solubility of poorly water-soluble drugs, and to increase their bioavailability and stability. In addition, cyclodextrins are used to reduce or prevent gastrointestinal or ocular irritation, reduce or eliminate unpleasant smells or tastes, prevent drug-drug or drug-additive interactions, or even to convert oils and liquid drugs into microcrystalline or amorphous powders.

Although the BTC compounds are soluble in water, the use of the cyclodextrins can increase that solubility even more so that a smaller volume of drug solution can be administered for a given dosage.

There are a number of cyclodextrins that can be used with the Compounds of the Invention. See for example, U.S. Pat. No. 4,727,064, hereby incorporated by reference in its entirety. Advantageous cyclodextrins are γ-cyclodextrin, 2-hydroxylpropyl-γ-cyclodextrin and 2-hydroxylpropyl-β-cyclodextrin, or other cyclodextrins which enhance the solubility of the BTC.

The use of gamma-cyclodextrin with TSC increases the solubility of TSC in water by 3-7 times. Although this is not as large a factor as seen in some other cases for increasing the solubility of an active agent with a cyclodextrin, it is important in allowing for the parenteral administration of TSC in smaller volume dosages to humans (or animals). Dosages of TSC and gamma-cyclodextrin have resulted in aqueous solutions containing as much as 44 milligrams of TSC per ml of solution, with an advantageous range of 20-30 mg/ml of solution. The solutions need not be equal-molar. The incorporation of the gamma cyclodextrin also allows for TSC to be absorbed into the blood stream when injected intramuscularly. Absorption is quick, and efficacious blood levels of TSC are reached quickly (as shown in rats).

The cyclodextrin formulation can be used with other trans carotenoids and carotenoid salts. The subject invention also includes novel compositions of carotenoids which are not salts (e.g. acid forms such as crocetin, crocin or the intermediate compounds noted above) and a cyclodextrin. In other words, trans carotenoids which are not salts can be formulated with a cyclodextrin. Mannitol can be added for osmolality, or the cyclodextrin BTC mixture can be added to isotonic saline (see below).

The amount of the cyclodextrin used is that amount which will contain the trans carotenoid but not so much that it will not release the trans carotenoid. Advantageously, the ratio of cyclodextrin to BTC, e.g., TSC, is 4 to 1 or 5 to 1. See also U.S. Patent Application No. 61/350,804, the content of which is hereby incorporated by reference in its entirety.

Cyclodextrin-Mannitol

A trans carotenoid such as TSC can be formulated with a cyclodextrin as noted above and a non-metabolized sugar such as mannitol (e.g. d-mannitol to adjust the osmotic pressure to be the same as that of blood). Solutions containing over 20 mg TSC/ml of solution can be made this way. This solution can be added to isotonic saline or to other isotonic solutions in order to dilute it and still maintain the proper osmolality.

Mannitol/Acetic Acid

A BTCS such as TSC can be formulated with mannitol such as d-mannitol, and a mild buffering agent such as acetic acid or citric acid to adjust the pH. The pH of the solution should be around 8 to 8.5. It should be close to being an isotonic solution, and, as such, can be injected directly into the blood stream.

Water+Saline

A BTCS such as TSC can be dissolved in water (advantageously injectable water). This solution can then be diluted with water, normal saline, Ringer's lactate or phosphate buffer, and the resulting mixture either infused or injected.

Buffers

A buffer such as glycine, bicarbonate, or sodium carbonate can be added to the formulation at a level of about 50 mM for stability of the BCT such as TSC.

TSC and Gamma-Cyclodextrin

The ratio of TSC to cyclodextrin is based on TSC:cyclodextrin solubility data. For example, 20 mg/ml TSC, 8% gamma cyclodextrin, 50 mM glycine, 2.33% mannitol with pH 8.2+/−0.5, or 10 mg/ml TSC and 4% cyclodextrin, or 5 mg/ml and 2% cyclodextrin. The ratios of these ingredients can be altered somewhat, as is obvious to one skilled in this art.

Mannitol can be used to adjust osmolality and its concentration varies depending on the concentration of other ingredients. The glycine is held constant. TSC is more stable at higher pHs. pH of around 8.2+/−0.5 is required for stability and physiological compatibility. The use of glycine is compatible with lyophilization. Alternatively, the TSC and cyclodextrin is formulated using a 50 mM bicarbonate buffer in place of the glycine.

Endotoxin Removal of Gamma-Cyclodextrin

Commercially available pharmaceutical grade cyclodextrin has endotoxin levels that are incompatible with intravenous injection. The endotoxin levels must be reduced in order to use the cyclodextrin in a BTC formulation intended for intravenous injection.

Thrombolysis and Formulation of Thrombolytics

Thrombolysis typically involves the injection of a thrombolytic (clot-busting) drugs through an intravenous (IV) line, or through a long catheter that delivers drugs directly to the site of the blockage (catheter directed thrombolysis). Catheter directed thrombolysis is a percutaneous procedure used to dissolve blood clots (thrombus) by administering a lytic directly into the clot through a catheter.

Formulation of thrombolytics is well known to those skilled in the art. A thrombolytic such as tPA, is typically administered via IV injection. If a diffusion enhancing drug has been administered, the advantage of administration of a thrombolytic is highest within the first ninety minutes, but can extend up to 3, 4, 5, 6, 9 or even 12 hours after the start of symptoms. The thrombolytic can be administered as catheter based thrombolysis.

Thrombolytic and/or diffusion enhancing drugs also can be given in combination with intravenous heparin, or low molecular weight heparin, which are anticoagulant drugs. Heparin and warfarin are often used to inhibit the formation and growth of existing thrombi.

Kits and Dual Chamber Delivery Systems

The diffusion enhancing compound such as TSC can be lyophilized and put in a vial which can be part of a vial kit system which also includes a vial with diluent such as water for injection, and a syringe for administration.

Dual-chamber delivery systems allow reconstitution of the lyophilized diffusion enhancing compound directly inside the system be it a syringe or a cartridge. The lyophilized diffusion enhancing compound such as TSC is located in one chamber and the diluent (e.g. water for injection) in the other. The drug is reconstituted just before administration. It is a simple and controllable process completed in a few easy steps.

In one embodiment, the diffusion enhancing compound such as TSC is loaded in an auto-injector. An auto-injector (or auto-injector) is a medical device designed to deliver a dose of a particular drug. Most auto-injectors are spring-loaded syringes. By design, auto-injectors are easy to use and are intended for self-administration by patients, or administration by untrained personnel. The site of injection is typically the thigh or the buttocks. The auto-injector typically keeps the needle tip shielded prior to injection and also has a passive safety mechanism to prevent accidental firing (injection). Injection depth can be adjustable or fixed and a function for needle shield removal can be incorporated. Just by pressing a button, the syringe needle is automatically inserted and the drug is delivered.

Uses of the Compounds and Compositions of the Invention

The subject invention provides methods of rapid response to the treatment of human patients, suspected of having, or diagnosed as having, a thrombosis or an embolism. The invention relates to diffusion enhancing compounds and their use with embolectomy or thrombectomy (endovascular catheter based or surgical) for the treatment of disorders resulting from the formation of an embolus or thrombus—infarction such as a myocardial infarction or stroke (brain infarction). The methods of the subject invention, via early (typically pre-hospital) administration of a diffusion enhancing compound, reduce cell death in the penumbra (area surrounding an ischemic event such as thrombotic or embolic stroke). The methods of the subject invention include administration of a diffusion enhancing compound such as TSC within a short time period, advantageously within 6 hours (more advantageously 2 hours or 90 minutes or less) of first symptoms of the thrombosis or embolism. The diffusion enhancing compound is typically administered by emergency medical personnel (e.g. paramedics) either upon arrival at the location of the patient or on the ambulance on the way to the hospital, or at the emergency room.

The subject invention relates to the use of diffusion enhancing compounds with procedures that treat infarction or reduce ischemia such as embolectomy or thrombectomy. In other embodiments, the methods of the subject invention include the use of diffusion enhancing compounds with procedures, other than thrombectomy or embolectomy, that reduce ischemia, such as angioplasty, PCI, or catheter based thrombolysis.

Included in the invention are methods for the treatment of disorders resulting from the formation of an embolus or thrombus such as a myocardial infarction or stroke (brain infarction). A thrombectomy is the removal of a blood clot (thrombus). A blood clot or foreign body that has moved and lodged in a blood vessel is called an embolus. An embolectomy is the removal of an embolus.

A diffusion enhancing compound such as trans sodium crocetinate (TSC) can be administered within a short time of a patient having symptoms that may be (suspected to be) the result of a thrombosis, an embolism or hemorrhage. If the patient is subsequently determined to have a thrombosis or an embolism, an embolectomy or thrombectomy can be performed to reduce deficits associated with the condition.

Embolectomy and Thrombectomy

There are many types of embolectomy and thrombectomy, depending on the blood vessel needing treatment. The general types include:

1) Catheter-based procedures involve passing a small tube (catheter) through a small incision in the groin to the clot site. Special instruments are used to break up or remove the embolus or thrombus (clot). A mesh stent device is often placed in the blood vessel to support it and keep it open. Catheter based thrombectomy can involve a balloon. The catheter is inserted into the blood vessel through a clot. The balloon is then inflated to extract the clot from the vessel. Catheters can involve the aspiration/suction of blood clots. Saline jets can dislodge and remove the clot using the Bernoulli effect. Other types of thrombectomy disrupt the clot mechanically using clot retriever, snare-like device, ultrasonography technology, or laser based devices. Ultrasound devices using ultrasound waves emitted at low frequency to create a physical fragmentation of the thrombus, can also be used.

ii) Surgical embolectomy/thrombectomy—open surgery involves making an incision (in some cases large) in the area of the blood clot through the blood vessel to remove it. Surgical embolectomy/thrombectomy is not common, but may be the best choice for emergencies to save an organ and in some other cases.

Embolectomy or thrombectomy are used to treat many types of blood clots or foreign bodies in a blood vessel. An embolectomy or thrombectomy can be used to treat:
Ischemic Stroke/Cerebral Infarction, which occurs when a clot develops or lodges in an artery in the brain (ischemic stroke).
Heart Attack/Myocardial Infarction, which occurs when a clot develops or lodges in an artery that supplies the heart muscle. It causes heart muscle to die from lack of oxygen.
Blood clots in peripheral arteries, which are arteries that supply the abdomen, arms and legs with blood
Deep vein thrombosis (DVT), which is a clot that develops in a vein in the legs or pelvis. A DVT fragment can break off and travel through the body and cause blockage of a lung artery (pulmonary embolism)
Pulmonary embolism (PE)/Lung Infarction, which is a clot that lodges in an artery in the lung
Blood clots in grafts, including bypass grafts
Foreign bodies, material left in the body after surgery or a procedure
Splenic infarction which occurs when the splenic artery or one of its branches are occluded, for example by a blood clot.
Limb infarction is an infarction of an arm or leg.

Ischemic Stroke

Ischemic stroke (brain infarction) occurs when an artery to the brain is blocked. Fast identification of stroke and stroke severity are crucial. The penumbra is the area surrounding an ischemic event such as thrombotic or embolic stroke. Immediately following the event, blood flow and therefore oxygen transport is reduced locally, leading to hypoxia of the cells near the location of the original insult. This can lead to hypoxic cell death (infarction) and amplify the original damage from the ischemia; however, the penumbra area may remain viable for several hours after an ischemic event due to the collateral arteries that supply the penumbral zone.

A major concern is to protect the penumbra by increasing oxygen transport and delivery to cells in the danger zone, thereby limiting cell death. There is a high correlation between the extent of spontaneous neurological recovery and the volume of penumbra that escapes infarction. Saving the penumbra improves the clinical outcome.

Catheter Based Neurothrombectomy Devices

Several endovascular mechanical means of removing clots have been used. These neurothrombectomy devices include:
clot retrievers including stent retrievers and devices with a balloon that can pull out a clot
aspiration/suction devices including rheolytic devices
ultrasound based devices;
laser based devices, and
snare-like devices.

These devices (1) allow patients to avoid or reduce the use of pharmacologic thrombolysis, thereby minimizing the risk for intracerebral hemorrhage; (2) can be used beyond the short timeframe to which tPA is limited. Mechanical removal can be done within eight to 24 hours or even longer, depending on the clot location; (3) provide more rapid recanalization than thrombolytics; and (4) provide a treatment option for thrombi more resistant to thrombolytic, including fibrinolytic, breakdown.

Mechanical Thrombectomy for Ischemic Stroke

Mechanical thrombectomy is a very important endovascular procedure, Doctors remove a blood clot by sending a wired-caged device called a stent retriever or aspiration device to the site of the blocked blood vessel in the brain.

Aspiration Devices

Suction thrombectomy devices employ vacuum aspiration to remove occlusive clot in acute ischemic stroke. While manual aspiration of target thrombi can be performed through any microcatheter, progress in developing suction thrombectomy devices required a technical solution to the problem of clogging of aspiration tips, a common occurrence when applying suction through a bore small enough to fit within intracranial arteries. The Penumbra System overcomes this obstacle by adding an in bore separator wire with a bulbous tip that the operator continually advances and retracts, disrupting attached clot and pulling in thrombus ahead of the catheter.

Stent Retrievers

Resembling a tiny wire cage, the stent retriever is threaded through a catheter into a blood vessel in the groin, then guided up to the blocked artery in the brain. The cage then opens up and captures the clot (entangling it within the stent struts). Then the stent, along with the clot, is removed (withdrawn back into the delivery catheter), immediately allowing blood to begin flowing again to the brain. Special suction tubes may also be used. Advantageously, the procedure should be done within six hours of acute stroke symptoms, and typically after a patient receives tPA.

Examples of stent retriever devices are Trevo Stent Retriever (Stryker) and the Solitaire Stent Retriever System (Covidien). The first retrievable stent approved in the United States is the Solitaire (Covidien), and several others have already been approved in Europe, including Trevo (Stryker), Revive (Codman), MindFrame (MindFrame Inc.), ReStore (Reverse Medical), and Pulse (which combines a stent retriever and an aspiration device, Penumbra) and Embol Trapll (Cerenovus).

Rapid and safe recanalisation and reperfusion of brain is the key factor, rather than specific device or technique. There are multiple options available. In addition to the primary device, many supplementary devices and techniques are used, for example, balloon guide catheters, intermediate catheters, and suction pumps versus manual aspiration, etc.

In the subject invention, a diffusion enhancing compound such as TSC is administered to a patient having, or suspected of having, an ischemic stroke. If it is determined that the patient does have an ischemic stroke, thrombectomy or embolectomy (e.g. using an aspiration device or a stent retriever device on the patient) is performed on the patient.

The early use of a diffusion enhancing compound such as TSC within the first 6 hours, advantageously 2 hours or 90 minutes, more advantageously 60 minutes, or most advantageously 30 minutes, of the onset of stroke symptoms prior to performing a thrombectomy or embolectomy, achieves a better outcome than the use of thrombectomy or embolectomy alone. Advantageously, the thrombectomy or embolectomy (e.g. mechanical) is performed as soon as possible (advantageously within 6 hours) after it has been determined that the stroke is an ischemic stroke.

The use of a diffusion enhancing compound can increase the window of opportunity of utilizing thrombectomy or embolectomy later in order to treat ischemic strokes. Thus, if a diffusion enhancing compound such as TSC is given to a human within 2 hours, then a thrombectomy or embolectomy can be performed 9, 12 or even up to or greater than 24 hours after the first stroke symptoms.

A patient showing signs of an ischemic stroke should be given a diffusion enhancing compound such as TSC, e.g., by IV injection or infusion or IM, at a dosage in the range of 0.05-2.5 mg/kg, advantageously 0.1-2 mg/kg, or 0.15-0.35 mg/kg.

In one embodiment of the invention for the treatment of ischemic stroke, a thrombolytic agent, such as tPA is administered after the diffusion enhancing compound e.g. bipolar trans carotenoid, but prior to the thrombectomy or embolectomy. The tPA can be administered IV or by catheter directed thrombolysis.

If a diffusion enhancing drug has been administered, the advantage of administration of a thrombolytic is highest within the first ninety minutes. Thrombolytic drugs can be given in combination with intravenous heparin, or low molecular weight heparin, which are anticoagulant drugs.

Heart Attack/Myocardial Infarction

Acute coronary syndrome (ACS) is a syndrome (set of signs and symptoms) due to decreased blood flow in the coronary arteries such that part of the heart muscle is unable to function properly or dies. Acute coronary syndrome is commonly associated with three clinical manifestations: ST elevation myocardial infarction (STEMI, 30%), non ST elevation myocardial infarction (NSTEMI, 25%), or unstable angina (38%). These types are named according to the appearance of the electrocardiogram (ECG).

Myocardial infarction (MI), commonly known as a heart attack, occurs when the blood supply to part of the heart is interrupted causing some heart cells to die. Heart attacks usually develop as a result of coronary artery disease. If changes to a blood vessel lead to dissection, i.e. a tear in the inner wall of the vessel, platelet aggregation (clotting) is activated, which leads to the vessel becoming blocked. Cell death is most commonly due to occlusion (blockage) of a coronary artery following the rupture of a vulnerable atherosclerotic plaque, which is an unstable collection of lipids (like cholesterol) and white blood cells (especially macrophages) in the wall of an artery. The resulting ischemia (restriction in blood supply) and oxygen shortage, if left untreated for a sufficient period of time, can cause damage and/or death (infarction) of heart muscle tissue (myocardium).

Most heart attacks are diagnosed from the results of an electrocardiogram [ECG] and a blood test, which can confirm whether or not a heart attack is occurring and if so, how it is affecting the heart. An ECG is often done in the ambulance by paramedics to make a preliminary diagnosis of a heart attack. While the final diagnosis rests in the hands of doctors, that preliminary diagnosis is important because it allows paramedics to begin treatment immediately—pre-hospital—often while still in the patient's home or in the ambulance.

The classic sign of a heart attack on the ECG is known as ST elevation. This is the electrical signal produced by a damaged segment of the heart muscle, and heart attacks which display this are known as “STEMI”—ST-elevation myocardial infarction. If a heart attack occurs without this ECG signal, it is known as NSTEMI (the N standing for non-). Such heart attacks are usually diagnosed on the basis of blood tests such as troponin-T and other enzymes released by damaged heart cells. These can take a few hours to show up in the blood so it is common practice to admit patients with possible heart attacks for a period of hours to get a second blood test.

Treatment of an MI is time-critical.

In a STEMI, treatments attempt to restore blood flow to the heart, and include percutaneous coronary intervention (PCI—see below), where the arteries are pushed open and may be stented, or thrombolysis, where the blockage is removed using medications.

In an NSTEMI treatments include blood thinners such as heparin, with the additional use of PCI in those at high risk.

In people with blockages of multiple coronary arteries and diabetes, coronary artery bypass surgery (CABG) is typically recommended rather than angioplasty.

Percutaneous Coronary Intervention

Percutaneous coronary intervention (PCI) is a non-surgical catheter based endovascular method used to open narrowed arteries that supply heart muscle with blood (coronary arteries). Primary PCI is the urgent use of PCI in a patient with acute myocardial infarction (heart attack)

Percutaneous means “through unbroken skin.” Percutaneous coronary intervention is performed by inserting a catheter through the skin in the groin or arm into an artery. At the leading tip of this catheter are several different devices including a balloon and a stent. The catheter and its devices are threaded through the inside of the artery back into an area of coronary artery narrowing or blockage. The “I” in percutaneous coronary intervention is for “Intervention,” which means that even if the person is actively having a heart attack (myocardial infarction or MI), percutaneous coronary intervention can be used to intervene and stop the attack by opening up the narrow or blocked coronary artery. This allows blood to flow to the heart muscle.

The term balloon angioplasty is commonly used to describe percutaneous coronary intervention, which describes the inflation of a balloon within the coronary artery to crush the plaque into the walls of the artery. While balloon angioplasty is still done as a part of nearly all percutaneous coronary interventions, it is rarely the only procedure performed. Another procedure done during a percutaneous coronary intervention is implantation of a stent. At times the procedure is done with
Rotational atherectomy—devices that can cut out plaque
Laser atherectomy
Brachytherapy (use of radioactive source to inhibit restenosis)

The angioplasty procedure usually consists of most of the following steps and is performed by a team made up of physicians, physician assistants, nurse practitioners, nurses, radiographers, and endovascular specialists; all of whom have extensive and specialized training in these types of procedures.
1. Access into the femoral artery in the leg (or, less commonly, into the radial artery or brachial artery in the arm) is created by a device called an “introducer needle”. This procedure is often termed percutaneous access.
2. Once access into the artery is gained, a “sheath introducer” is placed in the opening to keep the artery open and control bleeding.
3. Through this sheath, a long, flexible, soft plastic tube called a “guiding catheter” is pushed. The tip of the guiding catheter is placed at the mouth of the coronary artery. The guiding catheter also allows for radio-opaque dyes (usually iodine-based) to be injected into the coronary artery, so that the disease state and location can be readily assessed using real time X-ray visualization.
4. During the X-ray visualization, the cardiologist estimates the size of the coronary artery and selects the type of balloon catheter and coronary guidewire that will be used during the case. Heparin (a “blood thinner” or medicine used to prevent the formation of clots) is given to maintain blood flow. Bivalirudin when used instead of heparin has a higher rate of myocardial infarction but lower rates of bleeding.
5. The coronary guidewire, which is an extremely thin wire with a radio-opaque flexible tip, is inserted through the guiding catheter and into the coronary artery. While visualizing again by real-time X-ray imaging, the cardiologist guides the wire through the coronary artery to the site of the stenosis or blockage. The tip of the wire is then passed across the blockage. The cardiologist controls the movement and direction of the guidewire by gently manipulating the end that sits outside the patient through twisting of the guidewire.
6. While the guidewire is in place, it now acts as the pathway to the stenosis. The tip of the angioplasty or balloon catheter is hollow and is then inserted at the back of the guidewire—thus the guidewire is now inside of the angioplasty catheter. The angioplasty catheter is gently pushed forward, until the deflated balloon is inside of the blockage.
7. The balloon is then inflated, and it compresses the atheromatous plaque and stretches the artery wall to expand.
8. If a stent was on the balloon, then it will be implanted (left behind) to support the new stretched open position of the artery from the inside.

Early mechanical intervention (primary PCI) should be performed as soon as possible for patients with clinical presentation of STEMI and who have persistent ST-segment elevation or new or presumed new left bundle branch block (LBBB). In addition, it is reasonable to consider an early reperfusion strategy for patients presenting after more than 12 hours, provided there is clinical and/or ECG evidence of ongoing ischemia, with primary PCI being the preferred method in this population. After the onset of acute myocardial ischemia in patients with STEMI, timely myocardial reperfusion using PCI can salvage viable myocardium, limit MI size, preserve LV systolic function, and prevent the onset of heart failure.

“PCI” as used herein encompasses balloons and stents (metal scaffolding expanded inside the artery lumen). In one embodiment, the clot is removed prior to PCI e.g. by aspiration.

Thrombectomy is useful for treating: ST segment elevation myocardial infarction (STEMI); non-ST segment elevation myocardial infarction (NSTEMI); and recanalization of chronic thrombotic occlusion.

The process of removing a blood clot from a coronary artery can generally be performed using one of two very different techniques:
aspiration catheter (typically used for smaller clots) which allows the aspiration of blood clots.
mechanical thrombectomy catheter (typically used for larger clots). Large clots are broken up into smaller pieces before being safely removed.

Aspiration Thrombectomy

In manual thrombectomy, the cardiologist uses a syringe attached to a tube to create suction to remove the clot from the artery.

For patients with heart attack, the invasiveness of the thrombectomy procedure has been reduced to such an extent that only a small incision has to be made in a blood vessel in order to advance the specialist instruments required all the way to the heart. Aspiration of the blood clots is then performed either immediately or, in the case of larger blood clots, once the clot has been broken down into smaller fragments. Thrombectomy has improved both the safety and effectiveness of treatment for heart attack and, as a result, forms part of routine clinical practice.

Manual thrombectomy is simple, and is generally considered safe when performed according to a standard technique, which includes avoiding balloon pre-dilatation, aspirating with initial antegrade advancement of the catheter, and performing multiple passages until disappearance of visible thrombus. The Export thrombus aspiration catheter (Medronic Vascular) is a monorail system consisting of a dual lumen one for advancement over the wire (upper lumen) and one for thrombus aspiration (lower large lumen), with a distal radiopaque tip marker and a proximal luer lock port attached to a syringe for application of hand-powered suction to remove thrombus.

Mechanical Thrombectomy

Rheolytic Thrombectomy

A rheolytic thrombectomy is a procedure designed to remove clots. A special pump delivers high-pressure saline to the tip of the catheter. This creates a vacuum that breaks the clot into fragments and suctions the fragments out of the artery. The procedure is often used if there are extensive clots, such as in the case of a heart attack or vein graft disease.

Frequently used mechanical thrombectomy devices are the Angiojet (Medrad Interventional/Possis, Medical, Minnesota), providing rheolytic thrombectomy (RT), and the X-Sizer system (eV3, Minnesota)—see below. With both devices, multiple passes across the lesion should be performed until optimal angiographic result.

Mechanical thrombectomy is achieved by injecting pressurized saline through a hypotube by the distal tip of the coronary catheter thereby leading to a low-pressure zone (Bernoulli effect). The latter fragments the thrombus and the resulting debris is aspirated back and removed.

Rotating Cutter Thrombectomy

A rotating cutter catheter system can improve epicardial flow and accelerate ST-segment resolution compared with conventional PCI alone. The X-SIZER device consists of a helical cutter rotated at 2,100 rpm, which entrains and macerates thrombus and soft plaques but not fibrocalcific tissue. The device is a two-lumen over-the-wire system (available diameters 1.5 and 2.0 mm) with a helical shape cutter at its distal tip. The cutter rotates at 2,100 rpm driven by a hand-held battery motor unit. One catheter lumen is connected to a 250-ml vacuum bottle, and aspirated debris is collected in an in-line filter. Two or more passages across the lesion from proximal to distal are performed by slowly advancing the activated catheter.

Treatment does not merely involve clot removal. Once the entire clot—or at least the majority of it—has been removed, the patient is then typically treated with balloon catheters and stents as per standard treatment procedures. The patient is also usually treated with anticoagulant medication.

In the subject invention, a diffusion enhancing compound such as a BTCS compounds (e.g. TSC), is administered to a patient having, or suspected of having, a myocardial infarction. If it is determined that the patient is having a myocardial infarction, PCI and/or a thrombectomy method noted above, is performed on the patient.

Typically, the diffusion enhancing compound is administered as soon as possible, e.g. within 6 hours, advantageously within 90 minutes, more advantageously within 60 minutes, and most advantageously within 30 minutes after the onset of MI symptoms. It can be administered even prior to the patient having an ECG. Advantageously PCI and/or thrombectomy is performed as soon as the presence and location of the thrombus has been determined.

If a diffusion enhancing compound such as TSC is given to a human within the first 6 hours after the onset of MI symptoms, advantageously within 2 hours, and most advantageously within 1 hour, then PCI and/or thrombectomy can be performed 9, 12 or even up to or more than 24 hours after the onset of symptoms.

A diffusion enhancing compound such as TSC can be administered by various routes. For example, the compound which can be formulated with other compounds, can be administered at the proper dosage as an intravenous injection or infusion, an intramuscular injection, or in an oral form. The IV injection route is an advantageous route for giving a diffusion enhancing compound such as TSC for myocardial infarction. A patient showing signs of a myocardial infarction should be given a diffusion enhancing compound such as TSC, e.g., by IV injection or infusion or IM, at a dosage in the range of 0.05-2.5 mg/kg, advantageously 0.1-2 mg/kg, or 0.15-0.35 mg/kg.

Optionally, a therapeutically effective amount, i.e. a clot dissolving amount, of the thrombolytic agent such as tPA, can also be administered prior to PCI or thrombectomy. Formulations of thrombolytics are well known to those skilled in the art. The thrombolytic is typically administered via IV injection or by catheter directed thrombolysis.

If a diffusion enhancing drug has been administered, the advantage of administration of a thrombolytic is highest within the first ninety minutes. Thrombolytic drugs can be given in combination with intravenous heparin, or low molecular weight heparin, which are anticoagulant drugs.

Deep Vein Thrombosis

Deep vein thrombosis (also known as deep-vein thrombosis or deep venous thrombosis) is the formation of a blood clot (“thrombus”) in a deep vein. It is a form of thrombophlebitis (inflammation of a vein with clot formation).

Deep vein thrombosis commonly affects the leg veins (such as the femoral vein or the popliteal vein) or the deep veins of the pelvis. Occasionally the veins of the arm are affected (if spontaneous, this is known as Paget-Schrötter disease).

The current standard catheter-directed thrombolysis treatment uses a clot-dissolving drug only and, although highly effective, can take two to four days to work. This increases the patients' risk of bleeding as well as increasing their stay in the ICU. Although the catheter-directed thrombolysis technique has been available for about a decade, many DVT patients don't receive it.

Many patients are treated with blood thinners alone which can help prevent a life threatening pulmonary embolism, but do not help dissolve the clot. Long-term studies show that fifty percent of people with leg DVT treated with blood thinners alone develop the sequela of DVT, known as post-thrombotic syndrome. Post-thrombotic syndrome is caused by a combination of damage to the vein valves, as well as blocked blood flow in the vein from residual thrombus (clot). This condition is characterized by chronic leg pain and swelling which can lead to skin thickening and ulceration. Post-thrombotic syndrome is a common complication of DVT that is under-recognized and potentially preventable if the clots are dissolved early, before permanent damage to the vein occurs. If these patients are treated within 14 days of the onset of symptoms, the technique is successful in clearing the clot.

Catheter Based Techniques for Clot Removal

Typically, the thrombectomy used for DVT is aspiration thrombectomy, mechanical thrombectomy, or laser thrombectomy.

The “rapid lysis” technique combines a clot-dissolving drug with a clot removal device, thus improving the breaking up and dissolving of the clot, which is then vacuumed out of the vein and into the catheter, nonsurgically clearing away the deep vein thrombosis (DVT). Blood flow is restored throughout the leg, resolving symptoms.

Although the body may eventually dissolve clots, in the time needed to do so, permanent damage to the vein may occur, causing permanent disability and pain. Clots in the larger veins will rarely clear on their own.

A combination technique often allows the interventional radiologists to break up the clot in one treatment. The treatment works on the largest, most difficult clots, allowing resolution of DVT quickly and safely while restoring blood flow in the vein. The treatment can reduce the length of a hospital stay, thus reducing costs.

The interventional radiologist uses imaging to guide a catheter and the device into the vein and advances it to the blood clot. The device then sprays a diluted clot-dissolving drug into the clot at high force, helping to break up the clot and deliver the drug to a larger surface area throughout the clot. This enables the drug to remove the clot more quickly and efficiently. A powerful saline jet within the device creates a vacuum that draws the clot into the catheter, thus removing it from the body as the catheter is withdrawn.

In the subject invention, a diffusion enhancing compound such as a BTCS compounds (e.g. TSC) is administered to a patient having, or suspected of having, deep vein thrombosis. If it is determined that the patient does have deep vein thrombosis, a thrombectomy or embolectomy is performed on the patient.

Typically, the compound is administered as soon as possible, e.g. within 24 hours, advantageously within 2 hours or 90 minutes, more advantageously 60 minutes, or most advantageously within 30 minutes after the onset of symptoms.

The use of a diffusion enhancing compound can increase the window of opportunity of utilizing thrombectomy or embolectomy later in order to treat DVT. Thus, if a diffusion enhancing compound such as TSC is given to a human within 2 hours of the onset of symptoms, then a thrombectomy or embolectomy can be performed 9, 12 or even up to or more than 24 hours after the onset of DVT symptoms.

A diffusion enhancing compound such as TSC can be administered by various routes. For example, the compound which can be formulated with other compounds (excipients), can be administered at the proper dosage as an intravenous injection or infusion, an intramuscular injection, or in an oral form. The IV injection route is an advantageous route for giving a diffusion enhancing compound such as TSC for deep vein thrombosis. A patient showing signs of a deep vein thrombosis should be given a diffusion enhancing compound such as TSC, e.g., by IV injection or infusion, IM, or orally, at a dosage in the range of 0.05-2.5 mg/kg, 0.1-2 mg/kg, or 0.15-0.35 mg/kg.

Optionally, a therapeutically effective amount, i.e. a clot dissolving amount, of the thrombolytic agent such as tPA, can also be administered. Formulation of thrombolytics are well known to those skilled in the art. A thrombolytic such as tPA, is typically administered via IV injection or by catheter directed thrombolysis.

If a diffusion enhancing drug has been administered, the advantage of administration of a thrombolytic is highest within the first ninety minutes. Thrombolytic drugs can be given in combination with intravenous heparin, or low molecular weight heparin, which are anticoagulant drugs.

Pulmonary Embolism

Pulmonary embolism (PE) is a blockage of the pulmonary artery or one of its branches, usually occurring when a deep vein thrombus (blood clot from a vein) becomes dislodged from its site of formation and travels, or embolizes, to the arterial blood supply of one of the lungs. This process is termed thromboembolism.

Despite limited data, catheter directed techniques have been practiced for several decades: the FDA approved the Greenfield suction catheter for PE treatment in 1969. Currently, numerous devices employing various mechanisms to eradicate thrombus in the pulmonary arteries (PAs) are used. These utilize fragmentation, aspiration, mechanical thrombectomy, thrombolysis or a combination of these methods. The treatment endpoint is reversal of hemodynamic instability for massive PE, reversal of RVD and normalization of PA pressure in submassive PE, measured on echocardiography.

Catheter-Mediated Thrombus Fragmentation

Several methods exist for pulmonary artery (PA) thrombus fragmentation. The most widely used method is thromboembolus fragmentation by manual rotation of a pigtail catheter in the main, right, or left PA. Using the rotating pigtail catheter method, one group achieved an average of 33% recanalization through the thrombosed vessel, with concomitant reduction in PA pressure and shock index.

Newer devices are now available, such as the 8-Fr mechanical aspiration Aspirex1 Aspiration Thrombectomy Catheter (Straub Medical, Switzerland), which, similar to the AngioJet (Possis Medical, Inc., Minneapolis, Minn., USA), employs the Bernoulli hemodynamic principle (see below). To use this device, a long 8-Fr introducer sheath is placed in the thrombus-laden PA. The Aspirex1 catheter can be placed over a 0.018-inch exchange-length wire into the proximal thrombus. The catheter is advanced over the wire in thrombectomy mode through the thrombus. This can be repeated several times with possible endpoints of improved hemodynamics or improvement in oxygenation. One study of 30 patients with massive and submassive PE reported a complete clearance rate of >90% in over 80% of patients, with improvement in right heart strain; there were two major (bradycardia) and four minor (two access site hematomas, and two hemoptysis) procedure-related complications.

Rheolytic Thrombectomy

Rheolytic thrombectomy involves thromboembolus fragmentation using a saline jet directed from the tip of the catheter with simultaneous emulsified thrombus removal via a separate channel. Available devices include the Amplatz thrombectomy device (Microvena, White Bear Lake, Minn., USA), the cordis hydrolyser hydrodynamics thrombectomy catheter (Cordis, Johnson and Johnson, Japan), the Oasis Thrombectomy System (Boston Scientific Corporation, Natick, Mass., USA), and the AngioJet Rapid Thrombectomy System. The AngioJet system employs Bernoulli hydrodynamics: high velocity moving fluid has low central pressure, creating a vacuum, with preferential movement of surrounding molecules into the center of the fluid. The AngioJet device rapidly infuses heparinized saline, up to 360 mph, via the catheter endhole, reducing local pressure to a −600 mmHg, extracting soft thrombus into the catheter via a distal sideport.

The AngioJet device has pulse spray and thrombectomy modes. Initially, the catheter can be moved slowly back and forth in the affected main or lobar PA in pulse spray mode to infuse low dose tissue plasminogen activator (tPA) (18-20 mg mixed in 100 mL normal saline) throughout the thrombus. Following a prolonged pause, in regular mode, the catheter is again advanced and retracted within the thrombus in thrombectomy mode to aspirate clot. Immediate post-thrombectomy repeat pulmonary angiography and pressure measurements can confirm treatment effectiveness.

Catheter-Mediated Thromboembolus Aspiration

Large bore catheters, such as the AngioVac device (Vortex Medical, Inc., Norwell, Mass., USA), are available but are hampered by bulky size requiring 24-Fr introducer sheath access, difficulty accessing the PA, and requirement of veno-venous bypass. The AngioVac may have special application for treating IVC thrombosis in the setting of PE and can result in resolution of significant thrombus burden captured in an extracorporeal filter using veno-venous bypass and cardiac pump.

Modern suction thrombectomy catheters, including the Pronto XL extraction catheter (Vascular Solutions, Minneapolis, Minn., USA), available in 10-, 12- and 14-Fr sizes, are reportedly effective in acute massive PE by reducing the visible thrombus or the mean PA pressure. Its pigtail shape can be used for thromboembolus extraction from the main PA, and the straight tip version from segmental PAs. The pigtail version can also be used like a rotational catheter to manually fragment thrombus, simultaneously suctioning clot via distal sideholes using a 60-mL syringe. This device is FDA-approved for peripheral application; similar to newer devices, like the FlowTreiver (Mari Medical, Irvine, Calif., USA) and Indigo (Penumbra Inc., Alameda, Calif., USA), it is investigational in the PA.

Catheter Directed Thrombolysis Via Infusion Catheters

Catheter directed thrombolysis is a percutaneous procedure used to dissolve blood clots (thrombus) by administering a lytic directly into the clot through a catheter. Catheter directed thrombolysis via multi-sidehole infusion catheters is the least technically challenging of the catheter approaches to PE.

Ultrasound-assisted thrombolytic infusion catheters achieve accelerated thrombolysis using ultrasound waves. The EndoWave System (EKOS corporation, Bothwell, Mass., USA) consists of a 5-Fr 106-cm long catheter containing microinfusion pores within the 6-50 cm treatment segment of the catheter that optimize the interface of thrombus with an ultrasound core wire that contains small transducers allowing for delivery of ultrasound waves to the thromboembolus. Following access to the PA and angiographic examination, exchange is made over a 0.035-inch guidewire for EndoWave catheters containing the ultrasound core wire. The catheter also contains a port for tPA infusion (e.g., 0.5 mg/hour per catheter if bilateral, or 1 mg/hour per unilateral catheter), a port for saline to cool heat generated by the ultrasound waves, and an interface cable connected to a control unit in order to deliver ultrasound waves. Typically, tPA administration is performed over 18-24 hours.

In the subject invention, a diffusion enhancing compound such as a BTCS compounds (e.g. TSC), is administered to the patient having, or suspected of having, a pulmonary embolism. If it is determined that the patient is having a pulmonary embolism, an embolectomy (e.g. aspiration embolectomy, laser embolectomy or mechanical embolectomy), is performed on the patient.

Typically, the compound is administered as soon as possible, e.g. within 24 hours, advantageously within 4 or 2 hours or 90 minutes, more advantageously 60 minutes, or most advantageously within 30 minutes after the onset of symptoms.

The use of a diffusion enhancing compound can increase the window of opportunity of utilizing thrombectomy or embolectomy later in order to treat pulmonary embolism. Thus, if a diffusion enhancing compound such as TSC is given to a human within 2 hours of the onset of symptoms, then a thrombectomy or embolectomy can be performed 9, 12 or even up to 24 hours or more after the onset of pulmonary embolism symptoms.

The compound can be administered by various routes. For example, the compound which can be formulated with other compounds, can be administered at the proper dosage as an intravenous injection or infusion, an intramuscular injection, or in an oral form. The IV injection route is an advantageous route for giving a diffusion enhancing compound such as TSC for pulmonary embolism since the patient may well be unconscious. A patient showing signs of a pulmonary embolism should be given a diffusion enhancing compound such as TSC, e.g., by IV injection or infusion, or IM, at a dosage in the range of 0.1-2 mg/kg, advantageously 0.15-0.35 mg/kg.

Optionally, a therapeutically effective amount, i.e. a clot dissolving amount, of the thrombolytic agent such as tPA, is administered. Formulation of thrombolytics are well known to those skilled in the art. A thrombolytic such as tPA, is typically administered via IV injection. Alternatively, catheter directed thrombolysis is used.

If a diffusion enhancing drug has been administered, the advantage of administration of a thrombolytic is highest within the first ninety minutes. Thrombolytic drugs can be given in combination with intravenous heparin, or low molecular weight heparin, which are anticoagulant drugs.

Acute Limb Ischemia and Blood Clots in Peripheral Arteries

Acute limb ischemia is different from critical limb ischemia. Acute limb ischemia is a sudden lack of blood flow to the limb, for example caused by an embolus whereas critical limb ischemia is a late sign of a progressive chronic disease. Acute limb ischemia is caused by embolism or thrombosis, or rarely by dissection or trauma. Thrombosis is usually caused by peripheral vascular disease (atherosclerotic disease that leads to blood vessel blockage), while an embolism is usually of cardiac origin.

Catheter Based Embolectomy

A primary intervention in acute limb ischemia is emergency embolectomy using a Fogarty Catheter, providing the limb is still viable within the 4-6 hour timeframe from onset of symptoms. Aspiration embolectomy is a rapid and effective way of removing thrombi in thromboembolic occlusions of the limb arteries below the inguinal ligament, as in leg infarction.

Alternatively, catheter based thrombolysis is used. Other options include a vascular bypass to route blood flow around the clot.

Another technique disrupts the clot mechanically using either saline jets or, more recently, ultrasound waves. Saline jets dislodge the clot using the Bernoulli effect. Ultrasound waves, emitted at low frequency, create a physical fragmentation of the thrombus.

In the subject invention, a diffusion enhancing compound such as a BTCS compounds (e.g. TSC), is administered to a patient having, or suspected of having, acute limb ischemia or a clot on a peripheral artery. If it is determined that the patient does have acute limb ischemia or a clot on a peripheral artery, a thrombectomy or an embolectomy, is performed on the patient.

Typically, the compound is administered as soon as possible, i.e. within 2 hours, advantageously within 90 minutes, more advantageously 60 minutes, or most advantageously within 30 minutes after onset of symptoms.

The use of a diffusion enhancing compound can increase the window of opportunity of utilizing thrombectomy or embolectomy later in order to treat acute limb ischemia. Thus, if a diffusion enhancing compound such as TSC is given to a human within 2 hours of the onset of symptoms, then a thrombectomy or embolectomy can be performed 9, 12 or even up to 24 hours after the onset of acute limb ischemia symptoms.

The compound can be administered by various routes. For example, the compound which can be formulated with other compounds, can be administered at the proper dosage as an intravenous injection or infusion, an intramuscular injection, or in an oral form. The IV injection route is an advantageous route for giving a diffusion enhancing compound such as TSC for acute limb ischemia since the patient may well be unconscious. A patient showing signs of acute limb ischemia should be given a diffusion enhancing compound such as TSC, e.g., by IV injection or infusion, IM, or orally, at a dosage in the range of 0.1-2 mg/kg, advantageously 0.15-0.35 mg/kg.

Optionally, a thrombolytic (e.g. tPA) is administered prior to the thrombectomy or embolectomy. In one embodiment, catheter directed thrombolysis is used prior to the thrombectomy or embolectomy.

If a diffusion enhancing drug has been administered, the advantage of administration of a thrombolytic is highest within the first ninety minutes of the onset of symptoms. Thrombolytic drugs can be given in combination with intravenous heparin, or low molecular weight heparin, which are anticoagulant drugs.

Mesenteric Ischemia

Mesenteric ischemia is a medical condition in which injury of the small intestine occurs due to not enough blood supply. It can come on suddenly, known as acute mesenteric ischemia, or gradually, known as chronic mesenteric ischemia.

Embolectomy can also be used for mesenteric ischemia. Treatment of acute ischemia may include stenting or medications to break down the clot provided at the site of obstruction by interventional radiology.

In the subject invention, a diffusion enhancing compound such as a BTCS compounds (e.g. TSC), is administered to a patient having, or suspected of having, mesenteric ischemia. If it is determined that the patient does have mesenteric ischemia, a thrombectomy or an embolectomy, is performed on the patient.

Typically, the compound is administered as soon as possible, i.e. within 4 hours, advantageously within 2 hours or 90 minutes, more advantageously 60 minutes, or most advantageously within 30 minutes after the onset of symptoms.

The use of a diffusion enhancing compound can increase the window of opportunity of utilizing thrombectomy or embolectomy later in order to treat mesenteric ischemia. Thus, if a diffusion enhancing compound such as TSC is given to a human within 2 hours, then a thrombectomy or embolectomy can be performed 9, 12 or even up to 24 hours after the onset of mesenteric ischemia symptoms.

The compound can be administered by various routes. For example, the compound which can be formulated with other compounds, can be administered at the proper dosage as an intravenous injection or infusion, an intramuscular injection, or in an oral form. The IV injection route is an advantageous route for giving a diffusion enhancing compound such as TSC for mesenteric ischemia. A patient showing signs (symptoms) of mesenteric ischemia should be given a diffusion enhancing compound such as TSC, e.g., by IV injection or infusion, IM, or orally, at a dosage in the range of 0.1-2 mg/kg.

Optionally, a thrombolytic (e.g. tPA) is administered prior to the thrombectomy or embolectomy. In one embodiment, catheter directed thrombolysis is used prior to the thrombectomy or embolectomy.

If a diffusion enhancing drug has been administered, the advantage of administration of a thrombolytic is highest within the first ninety minutes. Thrombolytic drugs can be given in combination with intravenous heparin, or low molecular weight heparin, which are anticoagulant drugs.

Arterial Stenosis

Atherosclerosis is the most common cause of arterial narrowing (stenosis). The formation of atheromatous plaques within the wall of the artery bulges into the lumen and partially reduces blood flow to target organs. Atherosclerosis is progressive—it develops slowly over time. If left untreated, the plaque can grow to a size that significantly impairs the flow of blood leading to ischemia. In addition, rupture of the plaque and the formation of a blood clot may then completely occlude the artery. An embolus or thrombus may occur suddenly and the signs and symptoms of the occlusion may be evident within seconds or minutes, depending on the site. Depending on the size of the embolus or thrombus, the flow of blood may be partially or completely blocked.

Atherosclerosis develops within the wall of the artery while an embolus or thrombus develops inside the lumen of the artery. Atherosclerosis is more likely to affect large to medium sized arteries whereas and embolus or thrombus tends to cause a significant blockage in medium to small arteries.

Procedures for ischemia due to stenosis, even where an embolus or thrombus has not yet developed include:

Angioplasty

Balloon angioplasty employs a deflated balloon-tipped narrow catheter that is inserted through the skin of the groin or arm into an artery. The catheter is threaded through the artery until it arrives in the artery where there is narrowing. The catheter tip is then inserted through the narrowed area. Once in the narrowed area, the balloon is inflated, mashing the plaque into the vessel walls to reduce the narrowing. The balloon is then deflated and the catheter removed. The process is viewed by injecting a dye that allows the doctor to view the flowing blood as it goes through the arteries. This viewing method (angiogram) can be used to assure that the artery has increased blood flow after the balloon is deflated and removed.

Coronary Angioplasty

A coronary angiogram (an X-ray with radio-opaque contrast in the coronary arteries) that shows the left coronary circulation. The distal left main coronary artery (LMCA) is in the left upper quadrant of the image. Its main branches (also visible) are the left circumflex artery (LCX), which courses top-to-bottom initially and then toward the center-bottom, and the left anterior descending (LAD) artery, which courses from left-to-right on the image and then courses down the middle of the image to project underneath the distal LCX. The LAD, as is usual, has two large diagonal branches, which arise at the center-top of the image and course toward the center-right of the image.

A coronary angioplasty is a therapeutic procedure to treat the stenotic (narrowed) coronary arteries of the heart found in coronary heart disease. These stenotic segments are due to the buildup of cholesterol-laden plaques that form due to atherosclerosis.

Although treatment of acute heart attack is a very important use of PCI (see discussion of MI above), it has several other uses. Percutaneous coronary intervention can be used to relieve or reduce angina, prevent heart attacks, alleviate congestive heart failure, and allows some patients to avoid surgical treatment (coronary artery bypass graft or CABG) that involves extensive surgery and often long rehabilitation time.

Peripheral Angioplasty

Peripheral angioplasty refers to the use of a balloon to open a blood vessel outside the coronary arteries. It is commonly done to treat atherosclerotic narrowings of the abdomen, leg and renal arteries caused by peripheral artery disease. Often, peripheral angioplasty is used in conjunction with guide wire, peripheral stenting and an atherectomy.

Carotid Angioplasty

Carotid artery stenosis is treated with angioplasty in a procedure called carotid stenting for patients at high risk for carotid endarterectomy.

Renal Artery Angioplasty

Atherosclerotic obstruction of the renal artery can be treated with angioplasty with or without stenting of the renal artery. Renal artery stenosis can lead to hypertension and loss of renal function.

Venous Angioplasty

Angioplasty is occasionally used to treat venous stenosis, such as stenosis of the subclavian vein caused by thoracic outlet syndrome.

Endarterectomy Endarterectomy is a surgical procedure to remove the atheromatous plaque material, or blockage, in the lining of an artery constricted by the buildup of deposits. It is carried out by separating the plaque from the arterial wall.

The procedure is widely used on the carotid artery of the neck as a way to reduce the risk of stroke, particularly when the carotid artery is narrowed.

Atherectomy

Atherectomy is a minimally invasive endovascular surgery technique for removing atherosclerosis from blood vessels within the body. It is an alternative to angioplasty for the treatment of peripheral artery disease.

Coronary Artery Bypass Surgery

Coronary artery bypass surgery, also known as coronary artery bypass graft (CABG) surgery, and as heart bypass or bypass surgery, is a surgical procedure to restore normal blood flow to an obstructed coronary artery. A normal coronary artery transports blood to and from the heart muscle itself, not through the main circulatory system. There are two main approaches. In one, the left internal thoracic artery (internal mammary artery) is diverted to the left anterior descending branch of the left coronary artery. In the other, a great saphenous vein is removed from a leg; one end is attached to the aorta or one of its major branches, and the other end is attached to the obstructed artery immediately after the obstruction to restore blood flow.

A diffusion enhancing compound such as a BTCS compounds (e.g. TSC), can be used in conjunction with (typically prior to) each of the above procedures (e.g. angioplasty, PCI, CABG, atherectomy, endarterectomy) for arterial stenosis where a thrombus or embolus has not yet formed, to prevent heart attack, pulmonary embolism, or stroke (or other thrombosis or embolism), relieve or reduce angina or limb pain, alleviate congestive heart failure, and for angioplasty or PCI, allows some patients to avoid surgical treatment (coronary artery bypass graft or CABG).

The diffusion enhancing compound can be administered by various routes. For example, the compound (which can be formulated with other compounds), can be administered at the proper dosage as an intravenous injection or infusion, an intramuscular injection, or in an oral form. The IV injection route is an advantageous route for giving a diffusion enhancing compound such as TSC. The patient can be given a diffusion enhancing compound such as TSC, e.g., by IV injection or infusion, IM, or orally, 1-2 hours prior to the procedure at a dosage in the range of 0.05-2.5 mg/kg or 0.1-2 mg/kg.

It will be readily apparent to those skilled in the art that numerous modifications and additions can be made to both the present compounds and compositions, and the related methods without departing from the invention disclosed.

What is claimed is:

1. A method of treating a patient suspected of having an embolism or thrombosis comprising:
a) administering a diffusion enhancing compound to said patient within 2 hours of the onset of symptoms of an embolism or thrombosis,
b) determining whether said patient has an embolism or thrombosis, and if so determined,
c) performing an embolectomy or thrombectomy on said patient.

2. A method of treating a patient suspected of having an ischemic stroke comprising:
a) administering a diffusion enhancing compound to said patient within 2 hours of the onset of symptoms of the stroke,
b) determining whether said patient has an ischemic stroke, and if so determined,
c) performing an embolectomy or thrombectomy on said patient.

3. A method of treating a patient having a stroke where it is unknown whether the stroke is an ischemic stroke or a hemorrhagic stroke comprising:
a) administering a diffusion enhancing compound to said patient within 2 hours of the onset of symptoms of the stroke,
b) determining whether the stroke is an ischemic stroke, and if so determined,
c) performing an embolectomy or thrombectomy on said patient.

4. A method as in claim 2 or 3 wherein said embolectomy or thrombectomy is performed with a stent retriever.

5. A method as in claim 2 or 3 wherein said embolectomy or thrombectomy is performed with an aspiration device.

6. A method of treating a patient suspected of having a myocardial infarction comprising:
a) administering a diffusion enhancing compound to said patient within 2 hours of the onset of symptoms of a myocardial infarction,
b) determining whether said patient has a myocardial infarction, and if so determined,
c) performing a thrombectomy on said patient.

7. A method as in claim 6 further comprising performing a thrombolectomy prior to step c).

8. A method as in claim 6 wherein said thrombectomy is aspiration thrombectomy.

9. A method as in claim 6 wherein the myocardial infarction is STEMI.

10. A method of treating a patient suspected of having a pulmonary embolism comprising:
a) administering a diffusion enhancing compound to said patient within 2 hours of the onset of pulmonary embolism symptoms,
b) determining whether said patient has a pulmonary embolism, and if so determined,
c) performing an embolectomy on said patient.

11. A method of treating a patient suspected of having a deep vein thrombosis comprising:
a) administering a diffusion enhancing compound to said patient within 2 hours of the onset of deep vein thrombosis symptoms,
b) determining whether said patient has a deep vein thrombosis, and if so determined,
c) performing a thrombectomy on said patient.

12. A method of treating a patient suspected of having a blood clot in a peripheral artery comprising:
a) administering a diffusion enhancing compound to said patient within 2 hours of the onset of blood clot in a peripheral artery symptoms,
b) determining whether said patient has a blood clot in a peripheral artery, and if so determined,
c) performing a thrombectomy on said patient.

13. A method of reducing cell death in the penumbra of an embolism in a patient comprising:
a) administering a diffusion enhancing compound to said patient within 2 hours of the first symptom of the embolism, and
b) performing a thrombectomy or an embolectomy on said patient.

14. A method as in claim 13 wherein the penumbra is in the brain or heart.

15. A method of treating a patient suspected of having a myocardial infarction comprising:
a) administering a diffusion enhancing compound to said patient within 2 hours of the onset of MI symptoms,
b) determining whether said patient has a myocardial infarction, and if so determined,
c) performing PCI on said patient.

16. A method as in claim 15 wherein the clot is removed prior to performing PCI.

17. A method as in claim 15 wherein the PCI includes rotational or laser atherectomy, and/or brachytherapy.

18. A method of treating a patient having a hemorrhagic stroke comprising:
a) administering a diffusion enhancing compound to said patient within 2 hours of the first stroke symptoms, and
b) inserting a coil or clipping the artery at the site of the hemorrhage in said patient.

19. A method as in claim 1-13 wherein said embolectomy or thrombectomy is a catheter based endovascular embolectomy or thrombectomy.

20. A method as in claim 1-13 wherein said embolectomy or thrombectomy is performed by a balloon device or aspiration device.

21. A method as in claim 1-13 wherein said administration in step a) is within 90 or 60 minutes of the onset of symptoms.

22. A method as in claim 1-13 wherein said diffusion enhancing compound is a bipolar trans carotenoid salt.

23. A method as in claim 1-13 wherein said bipolar trans carotenoid salt is formulated with a cyclodextrin.

24. A method as in claim 1-13 wherein said diffusion enhancing compound is TSC.

25. A method as in claim 1-13 wherein the diffusion enhancing compound is administered IV or IM.

26. A method as in claim 1-13 wherein the diffusion enhancing compound is TSC and is administered at a dose of 0.2-2 mg/kg.

27. A method as in claim 1-12 further comprising the administration of a thrombolytic agent prior to step c).

28. A method as in claim 1-12 wherein said thrombolytic agent is selected from the group consisting of tPA, reteplase, tenecteplase, anistreplase, streptokinase, and urokinase.

29. A kit comprising:
a) a container comprising a diffusion enhancing compound, and
b) instructions for using the diffusion enhancing compound to treat a patient having or suspected of having, a thrombosis or embolism or a hemorrhage by administering the diffusion enhancing compound at a dose of 0.05-2.5 mg/kg.
